(19) United States
(12) Patent Application Publication
(10) Pub. No.: US 2026/0015356 A1
(43) Pub. Date: Jan. 15, 2026

(54) ADENOSINE RECEPTOR ANTAGONISTS AND USES THEREOF

(71) Applicant: Teon Therapeutics, Inc., Redwood City, CA (US)

(72) Inventors: Jiwen Liu, Redwood City, CA (US); Elfatih Elzein, Redwood City, CA (US)

(21) Appl. No.: 18/993,954

(22) PCT Filed: Jul. 11, 2023
(86) PCT No.: PCT/US2023/027387

§ 371 Date: Jan. 13, 2025

Related U.S. Application Data
(60) Provisional application No. 63/389,111, filed on Jul. 14, 2022.

(51) Int. Cl. C07D 473/06; A61K 31/52; A61K 31/675; A61K 45/06; C07F 9/6561

(52) U.S. Cl. CPC C07D 473/06; A61K 31/52; A61K 31/675; A61K 45/06; C07F 9/65616

(57) ABSTRACT

The present disclosure relates generally to adenosine receptor modulator compounds of formula (I), and methods of using such compounds in the treatment of conditions, diseases, or disorders that would benefit from modulation of A_{2B} adenosine receptor activity.

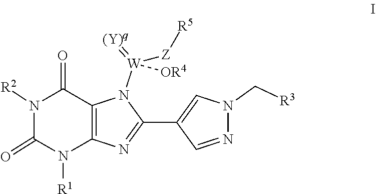

### CROSS-REFERENCE TO RELATED APPLICATIONS

This application claims the benefit under 35 U.S.C. § 119(e) of U.S. Provisional Application Ser. No. 63/389,111 filed Jul. 14, 2022, the contents of which are hereby incorporated by reference in its entirety.

### FIELD

Described herein are adenosine receptor modulator compounds, methods of making such compounds, pharmaceutical compositions and medicaments comprising such compounds, and methods of using such compounds in the treatment of conditions, diseases, or disorders that would benefit from modulation of A_{2R} adenosine receptor activity.

### BACKGROUND

Adenosine, an endogenous nucleoside, ubiquitously exists inside and outside of living cells. It plays multiple physiological roles to maintain the homeostasis of cells, tissues, and organs. Adenosine can exert its biological effects by interacting with a family of adenosine receptors known as A₁, A_{2A}, A_{2B}, and A₃ adenosine receptors. At adenosine receptors mediate mechanisms of tissue protection, especially for cardioprotection. A_{2A} adenosine receptors modulate coronary vasodilation and cancer immunity. A_{2B} adenosine receptors play a role in signaling pathways.

Some A_{2B} adenosine receptor antagonists are relatively insoluble in aqueous media and/or difficult to formulate using conventional pharmaceutical excipients, and thus can be difficult to formulate in a manner that provides reproducible plasma levels of the compound in mammals, in particular humans. A need exists for improving the bioavailability of A_{2B} adenosine receptor antagonists.

### SUMMARY

The present disclosure, in one embodiment, provides a compound of Formula I, or a pharmaceutically acceptable salt or solvate thereof,

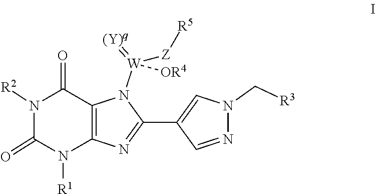

wherein R¹, R², R³, R⁴, R⁵, W, Z, Y, and q are as described in the detailed description.

The present disclosure, in one embodiment, provides compounds of Formula A or Formula B:

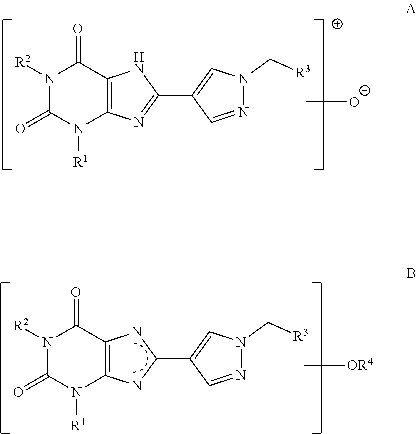

wherein R¹, R², R³, and R⁴, are as described in the detailed description.

### DETAILED DESCRIPTION

8-(1-(3-(trifluoromethyl)benzyl)-1H-pyrazol-4-yl)-3-ethyl-1-propyl-1H-purine-2,6(3H,7H)-dione (Compound A) is an A_{2B} adenosine receptor antagonist, which is a xanthine unsubstituted at the 7-position. It can be relatively insoluble in aqueous media and difficult to formulate using conventional pharmaceutical excipients, and thus can be difficult to formulate in a manner that provides reproducible plasma levels of the compound undergoing evaluation in mammals, in particular humans.

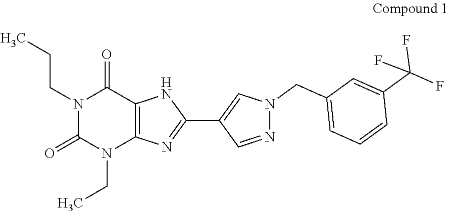 [SMILES: CCCn1c(=O)c2[nH]c(-c3cnn(Cc4cccc(C(F)(F)F)c4)c3)nc2n(CC)c1=O]

In some cases, prodrugs can be hydrolyzed by esterase (e.g., in gastrointestinal tract and/or in blood) and converted into Compound 1 in an aqueous solution. In some cases, acid labile prodrugs can be converted into Compound 1 in an acidic environment (e.g., in the stomach). In some cases, prodrugs, which are stable in the acidic environment and/or stable against hydrolysis by esterase, may not be a good prodrug candidate for Compound 1.

Accordingly, new prodrugs of the A_{2B} adenosine receptor antagonist can be developed to improve the formulation, pharmacokinetic profile, and/or bioavailability of the A_{2B} adenosine receptor antagonist Compound 1.

### Definitions

The following description sets forth exemplary embodiments of the present technology. It should be recognized, however, that such description is not intended as a limitation on the scope of the present disclosure but is instead provided as a description of exemplary embodiments.

As used in the present specification, the following words, phrases, and symbols are generally intended to have the meanings as set forth below, except to the extent that the context in which they are used indicates otherwise.

A dash (“-”) that is not between two letters or symbols is used to indicate a point of attachment for a substituent. For example, —C(O)NH₂ is attached through the carbon atom. A dash at the front or end of a chemical group is a matter of convenience; chemical groups may be depicted with or without one or more dashes without losing their ordinary meaning. A wavy line drawn through a line in a structure indicates a point of attachment of a group. Unless chemically or structurally required, no directionality is indicated or implied by the order in which a chemical group is written or named.

The prefix “C,” indicates that the following group has from u to v carbon atoms. For example, “C₁₋₆ alkyl” indicates that the alkyl group has from 1 to 6 carbon atoms.

Reference to “about” a value or parameter herein includes (and describes) embodiments that are directed to that value or parameter per se. In certain embodiments, the term “about” includes the indicated amount ±10%. In other embodiments, the term “about” includes the indicated amount ±5%. In certain other embodiments, the term “about” includes the indicated amount ±1%. Also, to the term “about X” includes description of “X”. Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

“Alkyl” refers to an unbranched or branched saturated hydrocarbon chain. As used herein, alkyl has 1 to 20 carbon atoms (i.e., C₁₋₂₀ alkyl), 1 to 8 carbon atoms (i.e., C₁₋₈ alkyl), 1 to 6 carbon atoms (i.e., C₁₋₆ alkyl), or 1 to 4 carbon atoms (i.e., C₁₋₄ alkyl). Examples of alkyl groups include methyl, ethyl, propyl, isopropyl, n-butyl, sec-butyl, iso-butyl, tert-butyl, pentyl, 2-pentyl, isopentyl, neopentyl, hexyl, 2-hexyl, 3-hexyl, and 3-methylpentyl. When an alkyl residue having a specific number of carbons is named by chemical name or identified by molecular formula, all positional isomers having that number of carbons may be encompassed; thus, for example, “butyl” includes n-butyl (i.e. —(CH₂)₃CH₃), sec-butyl (i.e. —CH(CH₃)CH₂CH₃), isobutyl (i.e. —CH₂CH(CH₃)₂) and tert-butyl (i.e. —C(CH₃)₃); and “propyl” includes n-propyl (i.e. —(CH₂)₂CH₃) and isopropyl (i.e. —CH(CH₃)₂.

“Alkenyl” refers to an alkyl group containing at least one carbon-carbon double bond and having from 2 to 20 carbon atoms (i.e., C₂₋₂₀ alkenyl), 2 to 8 carbon atoms (i.e., C₂₋₈ alkenyl), 2 to 6 carbon atoms (i.e., C₂₋₆ alkenyl), or 2 to 4 carbon atoms (i.e., C₂₋₄ alkenyl). Examples of alkenyl groups include ethenyl, propenyl, and butadienyl (including 1,2-butadienyl and 1,3-butadienyl).

“Alkynyl” refers to an alkyl group containing at least one carbon-carbon triple bond and having from 2 to 20 carbon atoms (i.e., C₂₋₂₀ alkynyl), 2 to 8 carbon atoms (i.e., C₂₋₈ alkynyl), 2 to 6 carbon atoms (i.e., C₂₋₆ alkynyl), or 2 to 4 carbon atoms (i.e., C₂₋₄ alkynyl). The term “alkynyl” also includes those groups having one triple bond and one double bond.

“Alkoxy” refers to the group “alkyl-O—”. Examples of alkoxy groups include methoxy, ethoxy, n-propoxy, iso-propoxy, n-butoxy, tert-butoxy, sec-butoxy, n-pentoxy, n-hexoxy, and 1,2-dimethylbutoxy.

“Haloalkoxy” refers to an alkoxy group as defined above, wherein one or more hydrogen atoms are replaced by a halogen.

“Alkylthio” refers to the group “alkyl-S—”.

“Alkylsulfone” refers to the group “alkyl-S(═O)₂—”.

“Alkylsulfoxide” refers to the group “alkyl-S(═O)—”.

“Acyl” refers to a group —C(O)R, wherein R is hydrogen, alkyl, cycloalkyl, heterocyclyl, aryl, heteroalkyl, or heteroaryl; each of which may be optionally substituted, as defined herein. Examples of acyl include formyl, acetyl, cylcohexylcarbonyl, cyclohexylmethyl-carbonyl, and benzoyl.

“Amido” refers to both a “C-amido” group which refers to the group —C(O)NR^{y}R^{z} and an “N-amido” group which refers to the group —NR^{y}C(O)R^{z}, wherein R^{y} and R^{z} are independently selected from the group consisting of hydrogen, alkyl, aryl, haloalkyl, or heteroaryl; each of which may be optionally substituted.

“Amino” refers to the group —NR^{y}R^{z} wherein R^{y} and R^{z} are independently selected from the group consisting of hydrogen, alkyl, haloalkyl, aryl, or heteroaryl; each of which may be optionally substituted.

“Amidino” refers to —C(NH)(NH₂).

“Aryl” refers to an aromatic carbocyclic group having a single ring (e.g. monocyclic) or multiple rings (e.g. bicyclic or tricyclic) including fused systems. As used herein, aryl has 6 to 20 ring carbon atoms (i.e., C₆₋₂₀ aryl), 6 to 12 carbon ring atoms (i.e., C₆₋₁₂ aryl), or 6 to 10 carbon ring atoms (i.e., C₆₋₁₀ aryl). Examples of aryl groups include phenyl, naphthyl, fluorenyl, and anthryl. Aryl, however, does not encompass or overlap in any way with heteroaryl defined below. If one or more aryl groups are fused with a heteroaryl, the resulting ring system is heteroaryl. If one or more aryl groups are fused with a heterocyclyl, the resulting ring system is heterocyclyl.

“Aryloxy” refers to the group “aryl-O—”.

“Arylthio” refers to the group “aryl-S—”.

“Arylsulfone” refers to the group “aryl-S(═O)₂—”.

“Arylsulfoxide” refers to the group “aryl-S(═O)—”.

“Carbamoyl” refers to both an “O-carbamoyl” group which refers to the group 4-C(O)NR_{y}R^{z} and an “N-carbamoyl” group which refers to the group —NR^{y}C(O)OR^{z}, wherein R^{y} and R^{z} are independently selected from the group consisting of hydrogen, alkyl, aryl, haloalkyl, or heteroaryl; each of which may be optionally substituted.

“Carboxyl” refers to —C(O)OH.

“Carboxyl ester” refers to both —OC(O)R and —C(O)OR, wherein R is hydrogen, alkyl, cycloalkyl, heterocyclyl, aryl, heteroalkyl, or heteroaryl; each of which may be optionally substituted, as defined herein.

“Cyano” or “carbonitrile” refers to the group —CN.

“Cycloalkyl” refers to a saturated or partially unsaturated cyclic alkyl group having a single ring or multiple rings including fused, bridged, and spiro ring systems. The term “cycloalkyl” includes cycloalkenyl groups (i.e. the cyclic group having at least one double bond). As used herein, cycloalkyl has from 3 to 20 ring carbon atoms (i.e., C₃₋₂₀ cycloalkyl), 3 to 12 ring carbon atoms (i.e., C₃₋₁₂ cycloalkyl), 3 to 10 ring carbon atoms (i.e., C₃₋₁₀ (cycloalkyl), 3 to 8 ring carbon atoms (i.e., C₃₋₈ cycloalkyl), or 3 to 6 ring carbon atoms (i.e., C₃₋₆ cycloalkyl). Examples of cycloalkyl groups include cyclopropyl, cyclobutyl, cyclopentyl, and cyclohexyl.

“Fluoroalkyl” refers to an alkyl group as defined above having at least one hydrogen replaced with a fluoro. In some embodiments, from 1 to 5 hydrogen atoms are replaced with a fluoro group.

“Fluoroalkoxy” refers to an alkoxy group as defined above having at least one hydrogen replaced with a fluoro. In some embodiments, from 1 to 5 hydrogen atoms are replaced with a fluoro group.

“Guanidino” refers to —NHC(NH)(NH₂).

“Imino” refers to a group —C(NR)R, wherein each R is alkyl, cycloalkyl, heterocyclyl, aryl, heteroalkyl, or heteroaryl; each of which may be optionally substituted, as defined herein.

“Halogen” or “halo” includes fluoro, chloro, bromo, and iodo. “Haloalkyl” refers to an unbranched or branched alkyl group as defined above, wherein one or more hydrogen atoms are replaced by a halogen. For example, where a residue is substituted with more than one halogen, it may be referred to by using a prefix corresponding to the number of halogen moieties attached. Dihaloalkyl and trihaloalkyl refer to alkyl substituted with two (“di”) or three (“tri”) halo groups, which may be, but are not necessarily, the same halogen. Examples of haloalkyl include difluoromethyl (—CHF₂) and trifluoromethyl (—CF₃).

“Heteroalkyl” refers to an alkyl group in which one or more of the carbon atoms (and any associated hydrogen atoms) are each independently replaced with the same or different heteroatomic group. The term “heteroalkyl” includes unbranched or branched saturated chain having carbon and heteroatoms. By way of example, 1, 2 or 3 carbon atoms may be independently replaced with the same or different heteroatomic group. Heteroatomic groups include, but are not limited to, —NR—, —O—, —S—, —S(O)—, —S(O)₂—, and the like, where R is H, alkyl, aryl, cycloalkyl, heteroalkyl, heteroaryl, or heterocyclyl, each of which may be optionally substituted. Examples of heteroalkyl groups include —OCH₃, —CH₂OCH₃, —SCH, —CH₂SCH₃, —NRCH₃, and —CH₂NRCH₃, where R is hydrogen, alkyl, aryl, arylalkyl, heteroalkyl, or heteroaryl, each of which may be optionally substituted. As used herein, heteroalkyl includes 1 to 10 carbon atoms, 1 to 8 carbon atoms, or 1 to 4 carbon atoms; and 1 to 3 heteroatoms, 1 to 2 heteroatoms, or 1 heteroatom.

“Heteroaryl” refers to an aromatic group having a single ring, multiple rings, or multiple fused rings, with one or more ring heteroatoms independently selected from nitrogen, oxygen, and sulfur. As used herein, heteroaryl includes 1 to 20 ring carbon atoms (i.e., C₁₋₂₀ heteroaryl), 3 to 12 ring carbon atoms (i.e., C₃₋₁₂ heteroaryl), or 3 to 8 carbon ring atoms (i.e., C₃₋₈ heteroaryl); and 1 to 5 heteroatoms, 1 to 4 heteroatoms, 1 to 3 ring heteroatoms, 1 to 2 ring heteroatoms, or 1 ring heteroatom independently selected from nitrogen, oxygen, and sulfur. Examples of heteroaryl groups include pyrimidinyl, purinyl, pyridyl, pyridazinyl, benzothiazolyl, and pyrazolyl. Examples of the fused-heteroaryl rings include, but are not limited to, benzo[d]thiazolyl, quinolinyl, isoquinolinyl, benzo[b]thiophenyl, indazolyl, benzo[d]imidazolyl, pyrazolo[1,5-a]pyridinyl, and imidazo[1,5-a]pyridinyl, where the heteroaryl can be bound via either ring of the fused system. Any aromatic ring, having a single or multiple fused rings, containing at least one heteroatom, is considered a heteroaryl regardless of the attachment to the remainder of the molecule (i.e., through any one of the fused rings). Heteroaryl does not encompass or overlap with aryl as defined above.

“Heterocyclyl” or “heterocyclcoalkyl” refers to a saturated or unsaturated cyclic alkyl group, with one or more ring heteroatoms independently selected from nitrogen, oxygen and sulfur. The term “heterocyclyl” includes heterocycloalkenyl groups (i.e. the heterocyclyl group having at least one double bond), bridged-heterocyclyl groups, fused-heterocyclyl groups, and spiro-heterocyclyl groups. A heterocyclyl may be a single ring or multiple rings wherein the multiple rings may be fused, bridged, or spiro. Any non-aromatic ring containing at least one heteroatom is considered a heterocyclyl, regardless of the attachment (i.e., can be bound through a carbon atom or a heteroatom). Further, the term heterocyclyl is intended to encompass any non-aromatic ring containing at least one heteroatom, which ring may be fused to an aryl or heteroaryl ring, regardless of the attachment to the remainder of the molecule. As used herein, heterocyclyl has 2 to 20 ring carbon atoms (i.e., C₂₋₂₀ heterocyclyl), 2 to 12 ring carbon atoms (i.e., C₂₋₁₂ heterocyclyl), 2 to 10 ring carbon atoms (i.e., C₂₋₁₀ heterocyclyl), 2 to 8 ring carbon atoms (i.e., C₂₋₈ heterocyclyl), 3 to 12 ring carbon atoms (i.e., C₃₋₁₂ heterocyclyl), 3 to 8 ring carbon atoms (i.e., C₃₋₈ heterocyclyl), or 3 to 6 ring carbon atoms (i.e., C₃₋₆ heterocyclyl); having 1 to 5 ring heteroatoms, 1 to 4 ring heteroatoms, 1 to 3 ring heteroatoms, 1 to 2 ring heteroatoms, or 1 ring heteroatom independently selected from nitrogen, sulfur or oxygen. Examples of heterocyclyl groups include pyrrolidinyl, piperidinyl, piperazinyl, oxetanyl, dioxolanyl, azetidinyl, and morpholinyl. As used herein, the term “bridged-heterocyclyl” refers to a four- to ten-membered cyclic moiety connected at two non-adjacent atoms of the heterocyclyl with one or more (e.g. 1 or 2) four- to ten-membered cyclic moiety having at least one heteroatom where each heteroatom is independently selected from nitrogen, oxygen, and sulfur. As used herein, bridged-heterocyclyl includes bicyclic and tricyclic ring systems. Also used herein, the term “spiro-heterocyclyl” refers to a ring system in which a three- to ten-membered heterocyclyl has one or more additional ring, wherein the one or more additional ring is three- to ten-membered cycloalkyl or three- to ten-membered heterocyclyl, where a single atom of the one or more additional ring is also an atom of the three- to ten-membered heterocyclyl. Examples of the spiro-heterocyclyl rings include bicyclic and tricyclic ring systems, such as 2-oxa-7-azaspiro[3.5]nonanyl, 2-oxa-6-azaspiro[3.4]octanyl, and 6-oxa-1-azaspiro[3.3]heptanyl. Examples of the fused-heterocyclyl rings include, but are not limited to, 1,2,3,4-tetrahydroisoquinolinyl, 4,5,6,7-tetrahydrothieno[2,3-c]pyridinyl, indolinyl, and isoindolinyl, where the heterocyclyl can be bound via either ring of the fused system.

“Hydroxy” or “hydroxyl” refers to the group —OH.

“One or more” means one, or one, or two, or three, or four, or five, or six.

“Oxo” refers to the group (═O) or (O).

“Nitro” refers to the group —NO₂.

“Sulfonyl” refers to the group —S(O)₂R, where R is alkyl, haloalkyl, heterocyclyl, cycloalkyl, heteroaryl, or aryl. Examples of sulfonyl are methylsulfonyl, ethylsulfonyl, phenylsulfonyl, and toluenesulfonyl.

“Alkylsulfonyl” refers to the group —S(O)₂R, where R is alkyl.

“Alkylsulfinyl” refers to the group —S(O)R, where R is alkyl.

“Thiocyanate” refers to the group —SCN.

“Thiol” refers to the group —SH.

“Thioxo” or “thione” refer to the group (=S) or (S).

Certain commonly used alternative chemical names may be used. For example, a divalent group such as a divalent “alkyl” group, a divalent “aryl” group, etc., may also be referred to as an “alkylene” group or an “alkylenyl” group, an “arylene” group or an “arylenyl” group, respectively. Also, unless indicated explicitly otherwise, where combinations of groups are referred to herein as one moiety, e.g. arylalkyl, the last mentioned group contains the atom by which the moiety is attached to the rest of the molecule.

The terms “optional” or “optionally” means that the subsequently described event or circumstance may or may not occur, and that the description includes instances where said event or circumstance occurs and instances in which it does not. Also, the term “optionally substituted” refers to any one or more hydrogen atoms on the designated atom or group may or may not be replaced by a moiety other than hydrogen.

Some of the compounds exist as tautomers. Tautomers are in equilibrium with one another. For example, amide containing compounds may exist in equilibrium with imidic acid tautomers. Regardless of which tautomer is shown, and regardless of the nature of the equilibrium among tautomers, the compounds are understood by one of ordinary skill in the art to comprise both amide and imidic acid tautomers. Thus, the amide containing compounds are understood to include their imidic acid tautomers. Likewise, the imidic acid containing compounds are understood to include their amide tautomers.

Any formula or structure given herein, is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine and chlorine, such as, but not limited to ²H (deuterium, D), ³H (tritium), ¹¹C, ¹³C, ¹⁴C, ¹⁵N, ¹⁸F, ³¹P, ³²P, ³⁵S, ³⁶Cl and ¹²⁵I. Various isotopically labeled compounds of the present disclosure, for example those into which radioactive isotopes such as ³H and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

The disclosure also includes “deuterated analogs” of compounds of Formula I or any subformula thereof in which from 1 to n hydrogens attached to a carbon atom is/are replaced by deuterium, in which n is the number of hydrogens in the molecule. Such compounds exhibit increased resistance to metabolism and are thus useful for increasing the half-life of any compound of Formula I when administered to a mammal, particularly a human. See, for example, Foster, “Deuterium Isotope Effects in Studies of Drug Metabolism,” Trends Pharmacol. Sci. 5(12):524-527 (1984). Such compounds are synthesized by means well known in the art, for example by employing starting materials in which one or more hydrogens have been replaced by deuterium.

Deuterium labelled or substituted therapeutic compounds of the disclosure may have improved DMPK (drug metabolism and pharmacokinetics) properties, relating to distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index. An ¹⁸F labeled compound may be useful for PET or SPECT studies. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent. It is understood that deuterium in this context is regarded as a substituent in the compound of Formula I.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen,” the position is understood to have hydrogen at its natural abundance isotopic composition. Accordingly, in the compounds of this disclosure any atom specifically designated as a deuterium (D) is meant to represent deuterium.

In many cases, the compounds of this disclosure are capable of forming acid and/or base salts by virtue of the presence of amino and/or carboxyl groups or groups similar thereto.

Provided are also pharmaceutically acceptable salts, hydrates, solvates, tautomeric forms, polymorphs, and prodrugs of the compounds described herein. “Pharmaceutically acceptable” or “physiologically acceptable” refer to compounds, salts, compositions, dosage forms and other materials which are useful in preparing a pharmaceutical composition that is suitable for veterinary or human pharmaceutical use.

The term “pharmaceutically acceptable salt” of a given compound refers to salts that retain the biological effectiveness and properties of the given compound, and which are not biologically or otherwise undesirable. “Pharmaceutically acceptable salts” or “physiologically acceptable salts” include, for example, salts with inorganic acids and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like. Likewise, pharmaceutically acceptable base addition salts can be prepared from inorganic and organic bases. Salts derived from inorganic bases include, by way of example only, sodium, potassium, lithium, ammonium, calcium and magnesium salts. Salts derived from organic bases include, but are not limited to, salts of primary, secondary and tertiary amines, such as alkyl amines (i.e., NH₂(alkyl)), dialkyl amines (i.e., HN(alkyl)₂), trialkyl amines (i.e., N(alkyl)₃), substituted alkyl amines (i.e., NH₂(substituted alkyl)), di(substituted alkyl) amines (i.e., HN(substituted alkyl)₂), tri(substituted alkyl) amines (i.e., N(substituted alkyl)₃), alkenyl amines (i.e., NH₂(alkenyl)), dialkenyl amines (i.e., HN(alkenyl)₂), trialkenyl amines (i.e., N(alkenyl)₃), substituted alkenyl amines (i.e., NH₂(substituted alkenyl)), di(substituted alkenyl) amines (i.e., HN(substituted alkenyl)₂), tri(substituted alkenyl) amines (i.e., N(substituted alkenyl)₃, mono-, di- or tri-cycloalkyl amines (i.e., NH₂(cycloalkyl), HN(cycloalkyl)₂, N(cycloalkyl)₃), mono-, di- or tri-arylamines (i.e., NH₂(aryl), HN(aryl)₂, N(aryl)₃), or mixed amines, etc. Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine, and the like.

The term “prodrug” refers to any compound that becomes an active form of a drug (e.g., Compound I) when administered to a subject, e.g., upon metabolic processing of the prodrug.

Prodrugs are often useful because, in some situations, they are easier to administer than the parent drug. They are, for instance, bioavailable by oral administration whereas the parent is not. Further or alternatively, the prodrug also has improved solubility in pharmaceutical compositions over the parent drug. In some embodiments, the design of a prodrug increases the effective water solubility. In certain embodiments, upon in vivo administration, a prodrug is chemically converted to the biologically, pharmaceutically or therapeutically active form of the compound. In certain embodiments, a prodrug is enzymatically metabolized by one or more steps or processes to the biologically, pharmaceutically or therapeutically active form of the compound.

Prodrugs of compound A described herein include, but are not limited to, compounds where the nitrogen atom is incorporated into an alkyl carbamate, (acyloxy)alkyl carbamate, acyloxyalkyl ester, alkoxycarbonyloxyalkyl ester, alkyl ester, aryl ester, phosphate ester, sugar ester, ether, N-acyloxyalkoxycarbonyl, N-acyloxyakyl, dihydropyridinepyridinium salt system (redox systems), (phosphoryloxy)methyl carbamate, (acyloxy)alkyl carbamate, and the like.

In some embodiments, prodrugs of compound A are formed by N-acyloxyalkylation, N-hydroxyalkylation, N-(phosphoryloxy)alkylation, N-acyloxyalkylation, N-hydroxyalkylation, N-(phosphoryloxy)alkylation, N-acylation (amides and carbamates), N-(oxodioxolenyl)methylation, and the like.

The term “substituted” means that any one or more hydrogen atoms on the designated atom or group is replaced with one or more substituents other than hydrogen, provided that the designated atom's normal valence is not exceeded. The one or more substituents include, but are not limited to, alkyl, alkenyl, alkynyl, alkoxy, acyl, amino, amido, amidino, aryl, azido, carbamoyl, carboxyl, carboxyl ester, cyano, guanidino, halo, haloalkyl, haloalkoxy, heteroalkyl, heteroaryl, heterocyclyl, hydroxy, hydrazino, imino, oxo, nitro, alkylsulfinyl, sulfonic acid, alkylsulfonyl, thiocyanate, thiol, thione, or combinations thereof. Polymers or similar indefinite structures arrived at by defining substituents with further substituents appended ad infinitum (e.g., a substituted aryl having a substituted alkyl which is itself substituted with a substituted aryl group, which is further substituted by a substituted heteroalkyl group, etc.) are not intended for inclusion herein. Unless otherwise noted, the maximum number of serial substitutions in compounds described herein is three. For example, serial substitutions of substituted aryl groups with two other substituted aryl groups are limited to ((substituted aryl)substituted aryl) substituted aryl. Similarly, the above definitions are not intended to include impermissible substitution patterns (e.g., methyl substituted with 5 fluorines or heteroaryl groups having two adjacent oxygen ring atoms). Such impermissible substitution patterns are well known to the skilled artisan. When used to modify a chemical group, the term “substituted” may describe other chemical groups defined herein. Unless specified otherwise, where a group is described as optionally substituted, any substituents of the group are themselves unsubstituted. For example, in some embodiments, the term “substituted alkyl” refers to an alkyl group having one or more substituents including hydroxyl, halo, alkoxy, cycloalkyl, heterocyclyl, aryl, and heteroaryl. In other embodiments, the one or more substituents may be further substituted with halo, alkyl, haloalkyl, hydroxyl, alkoxy, cycloalkyl, heterocyclyl, aryl, or heteroaryl, each of which is substituted. In other embodiments, the substituents may be further substituted with halo, alkyl, haloalkyl, alkoxy, hydroxyl, cycloalkyl, heterocyclyl, aryl, or heteroaryl, each of which is unsubstituted.

As used herein, “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

A “solvate” is formed by the interaction of a solvent and a compound. Solvates of salts of the compounds described herein are also provided. Hydrates of the compounds described herein are also provided.

### LIST OF ABBREVIATIONS AND ACRONYMS

### Abbreviation Meaning

° C. Degree Celsius
aq. Aqueous
DMF Dimethyl formamide
g Grams
hrs Hours
M Molar
mg Milligram
MHz Megahertz
ml/mL Milliliter
mM Millimolar
mmol Millimole
nL Nanoliter
nm Nanometer
μL/μl Microliter
μM Micromolar
THF Tetrahydrofuran

### COMPOUNDS

Provided herein are compounds of Formula I, or a pharmaceutically acceptable salt or solvate thereof,

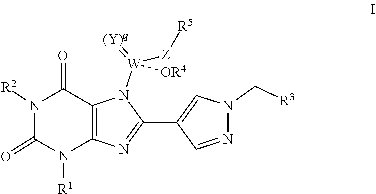

wherein
the dotted line - - indicates that the referenced group may be present or absent;
when W is C and Y is O, then q is 1, Z is absent, S, or —N—(R⁴)—, and OR⁴ is absent;
when W is C and Y is S, then q is 1, Z is absent, O, S, or —N—(R⁴)—, and OR⁴ is absent;
when W is C and Y is —N—(R⁴), then q is 1, Z is —CH₂— or —N—(R⁴)—, and OR⁴ is absent;
when W is P and Y is O, then q is 1, Z is O or —N—(R⁴)—, and OR⁴ is present;
when W is S and Y is O or —N—(R⁴), then q is 1 or 2, Z is —CH₂— or —N—(R⁴)—, and OR⁴ is absent;
R¹ and R² are each independently selected from hydrogen and substituted or unsubstituted C₁-C₆ alkyl;
R³ is selected from substituted or unsubstituted phenyl and substituted or unsubstituted heteroaryl, wherein if R³ is substituted then R³ is substituted with one or more groups selected from halogen, —CN, —OH, C₁-C₄alkyl, C₂-C₄alkenyl, C₂-C₄alkynyl, C₁-C₄alkoxy, C₁-C₄fluoroalkyl, C₁-C₄fluoroalkoxy, and substituted or unsubstituted C₁-C₄heteroalkyl; each R⁴ is independently hydrogen or C₁-C₆ alkyl;
R⁵ is hydrogen, R⁷, —C(═O)R⁷, —C(═O)—OR⁷, —C(═O)N(R⁷)(R⁸), —C(═O)—SR⁷, or —P(═O)(OR⁹)₂;
R⁷ is substituted or unsubstituted C₁-C₆ alkyl, substituted or unsubstituted heteroalkyl, substituted or unsubstituted C₃-C₁₀cycloalkyl, substituted or unsubstituted C₂-C₁₀heterocycloalkyl, substituted or unsubstituted phenyl, substituted or unsubstituted heteroaryl, -alkyl-(substituted or unsubstituted phenyl), -alkyl-(substituted or unsubstituted heteroaryl), -alkyl-(substituted or unsubstituted cycloalkyl), -alkyl-(substituted or unsubstituted heterocycloalkyl), —(C(R¹⁰)₂O)ₘ—R¹¹, —(CH₂CH₂O)ₙ—R¹¹, or —(C(R¹⁰)₂)ₚ—OR¹¹;
R⁸ is hydrogen or C₁-C₆ alkyl;
or R⁷ and R⁸ are taken together with the nitrogen atom to which they are attached to form a substituted or unsubstituted C₂-C₁₀heterocycloalkyl;
each R⁹ is independently selected from hydrogen and C₁-C₆ alkyl;
each R¹⁰ is independently selected from hydrogen and C₁-C₆ alkyl;
R¹¹ is hydrogen, substituted or unsubstituted C₁-C₆ alkyl, substituted or unsubstituted heteroalkyl, substituted or unsubstituted C₂-C₁₀heterocycloalkyl, —C(═O)R¹², —C(═O)—OR¹², —C(═O)N(R¹²)(R⁸), —C(═O)—SR¹², or —P(=O)(OR⁹)₂;
R¹² is hydrogen, substituted or unsubstituted C₁-C₆ alkyl, substituted or unsubstituted heteroalkyl, substituted or unsubstituted C₃-C₁₀cycloalkyl, substituted or unsubstituted C₂-C₁₀heterocycloalkyl, substituted or unsubstituted phenyl, substituted or unsubstituted heteroaryl, -alkyl-(substituted or unsubstituted phenyl), or -alkyl-(substituted or unsubstituted heteroaryl);
m is 1, 2, 3, 4, 5, or 6;
n is 1, 2, 3, 4, 5, or 6;
p is 1, 2, 3, 4, 5, or 6;
wherein substituted means that the referenced group is substituted with one or more additional groups individually and independently selected from halogen, —CN, —NH₂, —NH(C₁-C₆ alkyl), —N(alkyl)₂, —OH, —CO₂H, —CO₂ C1-C₆ alkyl, —C(═O)NH₂, —C(═O)NH(C₁-C₆ alkyl), —C(═O)N(C₁-C₆ alkyl)₂, —S(═O)₂NH₂, —S(═O)₂NH(alkyl), —S(═O)₂N(C₁-C₆ alkyl)₂, C₁-C₆ alkyl, cycloalkyl, fluoro C₁-C₆ alkyl, heteroalkyl, C₁-C₆ alkoxy, fluoro C₁-C₆ alkoxy, heterocycloalkyl, aryl, heteroaryl, aryloxy, C₁-C₆ alkylthio, arylthio, C₁-C₆ alkylsulfoxide, arylsulfoxide, C₁-C₆ alkylsulfone, and arylsulfone.

In some embodiments of Formula I is a compound, or a pharmaceutically acceptable salt or solvate thereof, having the structure of Formula Ia:

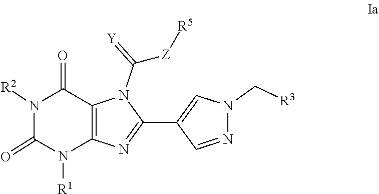

wherein
when Y is O, then Z is absent, S, or —N—(R⁴)—;
when Y is S, then Z is absent, O, S, or —N—(R⁴)—;
R¹ and R² are each independently selected from hydrogen and C₁-C₆ alkyl;
R³ is selected from substituted or unsubstituted phenyl, wherein if R³ is substituted then R³ is substituted with one or more groups selected from halogen, —CN, C₁-C₆ alkyl, and C₁-C₆ fluoroalkyl;
R⁴ is hydrogen or C₁-C₆ alkyl;
R⁵ is hydrogen, R⁷, —C(═O)R⁷, —C(═O)—OR⁷, —C(═O)N(R⁷)(R⁸), —C(═O)—SR⁷, —P(=O)(OR⁹)₂, —(C(R¹⁰)₂O)ₘ—R¹¹, —(CH₂CH₂O)ₙ—R₁₁, or —(C(R¹⁰)₂)ₚ—OR¹¹;
R⁷ is substituted or unsubstituted C₁-C₆ alkyl, substituted or unsubstituted hetero C₁-C₆ alkyl, substituted or unsubstituted C₃-C₁₀cycloalkyl, substituted or unsubstituted heterocycloalkyl, substituted or unsubstituted phenyl, substituted or unsubstituted heteroaryl, —C₁-C₆ alkyl-(substituted or unsubstituted phenyl), —C₁-C₆ alkyl-(substituted or unsubstituted heteroaryl), —C₁-C₆ alkyl-(substituted or unsubstituted C₃-C₁₀ cycloalkyl), or —C₁-C₆ alkyl-(substituted or unsubstituted heterocycloalkyl);
R⁸ is hydrogen or C₁-C₆ alkyl;
or R⁷ and R⁸ are taken together with the nitrogen atom to which they are attached to form a substituted or unsubstituted heterocycloalkyl;
each R⁹ is independently selected from hydrogen and C₁-C₆ alkyl;
each R¹⁰ is independently selected from hydrogen and C₁-C₆ alkyl;
R¹¹ is hydrogen, substituted or unsubstituted C₁-C₆ alkyl, substituted or unsubstituted heteroalkyl, substituted or unsubstituted heterocycloalkyl, —C(═O)R¹², —C(═O)—OR¹², —C(═O)N(R¹²)(R⁸), —C(═O)—SR¹², or —P(=O)(OR⁹)₂;
R¹² is hydrogen, substituted or unsubstituted C₁-C₆ alkyl, substituted or unsubstituted heteroalkyl, substituted or unsubstituted C₃—C o cycloalkyl, substituted or unsubstituted heterocycloalkyl, substituted or unsubstituted phenyl, substituted or unsubstituted heteroaryl, —C₁-C₆ alkyl-(substituted or unsubstituted phenyl), or —C₁-C₆ alkyl-(substituted or unsubstituted heteroaryl);
m is 1, 2, 3, 4, 5, or 6;
n is 1, 2, 3, 4, 5, or 6;
p is 1, 2, 3, 4, 5, or 6;
wherein substituted means that the referenced group is substituted with one or more additional groups individually and independently selected from halogen, —CN, —NH₂, —NH(C₁-C₆ alkyl), —N(C₁-C₆ alkyl)₂, —OH, —CO₂H, —CO₂—C₁-C₆ alkyl, —C(═O)NH₂, —C(═O)NH(C₁-C₆ alkyl), —C(═O)N(C₁-C₆ alkyl)₂, —S(═O)₂NH₂, —S(═O)₂NH(C₁-C₆ alkyl), —S(═O)₂N(C₁-C₆ alkyl)₂, C₁-C₆ alkyl, fluoroC₁-C₆ alkyl, C₁-C₆ alkoxy, fluoroC₁-C₆ alkoxy, C₁-C₆ alkylthio, C₁-C₆ alkylsulfoxide, and C₁-C₆ alkylsulfone.

In some embodiments, a compound of Formula I, or a pharmaceutically acceptable salt or solvate thereof, has the structure of Formula IIa:

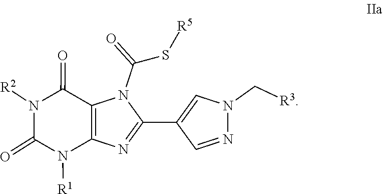

where R¹, R², R³, and R⁵ are as defined herein.

In some embodiments, a compound of Formula I, or a pharmaceutically acceptable salt or solvate thereof, has the structure of Formula IIb:

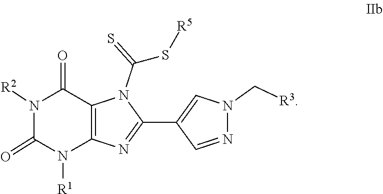

where R¹, R², R³, and R⁵ are as defined herein.

In some embodiments, a compound of Formula I, or a pharmaceutically acceptable salt or solvate thereof, has the structure of Formula IIIa:

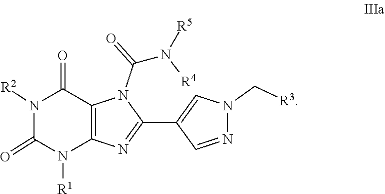

where R¹, R², R³, R⁴, and R⁵ are as defined herein.

In some embodiments, a compound of Formula I, or a pharmaceutically acceptable salt or solvate thereof, has the structure of Formula IIIb:

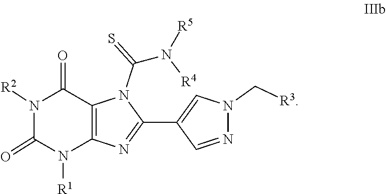

where R¹, R², R³, R⁴, and R⁵ are as defined herein.

In some embodiments, a compound of Formula I, or a pharmaceutically acceptable salt or solvate thereof, has the structure of Formula IVa:

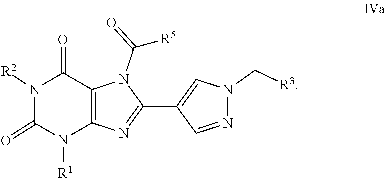

where R¹, R², R³, and R⁵ are as defined herein.

In some embodiments, a compound of Formula I, or a pharmaceutically acceptable salt or solvate thereof, has the structure of Formula IVb:

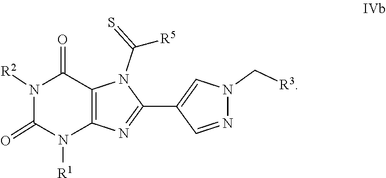

where R¹, R², R³, and R⁵ are as defined herein.

In some embodiments, a compound of Formula I, or a pharmaceutically acceptable salt or solvate thereof, has the structure of Formula Va:

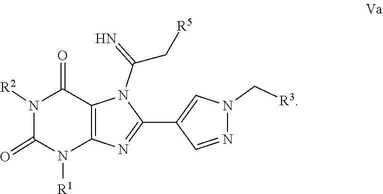

where R¹, R², R³, and R⁵ are as defined herein.

In some embodiments, a compound of Formula I, or a pharmaceutically acceptable salt or solvate thereof, has the structure of Formula Vb:

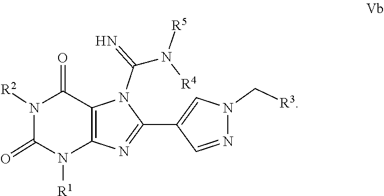

where R¹, R², R³, R⁴, and R⁵ are as defined herein.

In some embodiments, a compound of Formula I, or a pharmaceutically acceptable salt or solvate thereof, has the structure of Formula VIa:

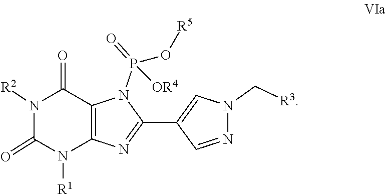

where R¹, R², R³, R⁴, and R⁵ are as defined herein.

In some embodiments, a compound of Formula I, or a pharmaceutically acceptable salt or solvate thereof, has the structure of Formula VIb:

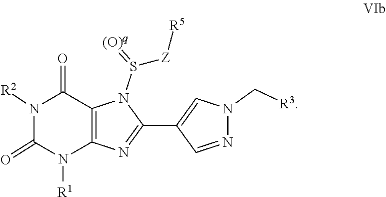

where q, Z, R¹, R², R³, and R⁵ are as defined herein.

In some embodiments, for any formula described herein, R¹ is methyl, ethyl, or n-propyl.

In some embodiments, for any formula described herein, R² is methyl, ethyl, or n-propyl

In some embodiments, for any formula described herein, R³ substituted or unsubstituted phenyl, wherein if R³ is substituted then R³ is substituted with one or more groups selected from halogen and C₁-C₆ fluoroalkyl.

In some embodiments, for any formula described herein, R³ is phenyl substituted with C₁-C₆ fluoroalkyl.

In some embodiments, for any formula described herein, each R⁴ is independently C₁-C₆ alkyl.

In some embodiments, for any formula described herein, R⁴ is —C(═O)R⁷, —C(═O)—OR⁷, —C(═O)N(R⁷)(R⁸), —C(═O)—SR⁷, —P(═O)(OR⁹)₂, —(C(R¹⁰)₂O)ₘ—R¹¹, —(CH₂CH₂O)ₙ—R¹¹, or —(C(R¹⁰)₂)ₚ—OR¹¹.

In some embodiments, for any formula described herein, R⁵ is substituted or unsubstituted C₁-C₆ alkyl, substituted or unsubstituted hetero C₁-C₆ alkyl, substituted or unsubstituted C₃-C₁₀cycloalkyl, substituted or unsubstituted heterocycloalkyl, substituted or unsubstituted phenyl, substituted or unsubstituted heteroaryl, —C₁-C₆ alkyl-(substituted or unsubstituted phenyl), —C₁-C₆ alkyl-(substituted or unsubstituted heteroaryl), —C₁-C₆ alkyl-(substituted or unsubstituted C₃-C₁₀ cycloalkyl), or —C₁-C₆ alkyl-(substituted or unsubstituted heterocycloalkyl).

In some embodiments, for any formula described herein, R⁵ is substituted or unsubstituted C₁-C₆ alkyl

In some embodiments, for any formula described herein, R⁵ is methyl, ethyl, n-propyl, isopropyl, n-butyl, or isobutyl.

In some embodiments, provided is a compound of Formula I or Ia, or a pharmaceutically acceptable salt or solvate thereof, selected from:

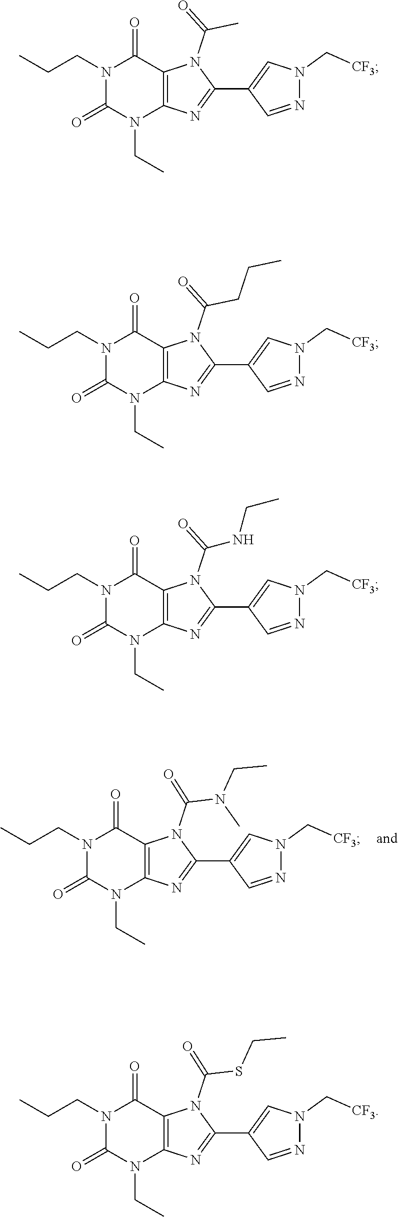 [SMILES: CCCn1c(=O)c2c(nc(-c3cnn(CC(F)(F)F)c3)n2C(C)=O)n(CC)c1=O ; CCCC(=O)n1c(-c2cnn(CC(F)(F)F)c2)nc2c1c(=O)n(CCC)c(=O)n2CC ; CCCn1c(=O)c2c(nc(-c3cnn(CC(F)(F)F)c3)n2C(=O)NCC)n(CC)c1=O ; CCCn1c(=O)c2c(nc(-c3cnn(CC(F)(F)F)c3)n2C(=O)N(C)CC)n(CC)c1=O ; CCCn1c(=O)c2c(nc(-c3cnn(CC(F)(F)F)c3)n2C(=O)SCC)n(CC)c1=O]

Provided herein is a compound of Formula A, or a pharmaceutically acceptable salt or solvate thereof,

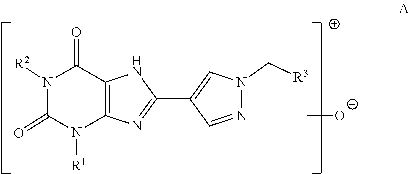

wherein
R¹ and R² are each independently selected from hydrogen and substituted or unsubstituted C₁-C₆ alkyl; and
R³ is selected from substituted or unsubstituted phenyl and substituted or unsubstituted heteroaryl, wherein if R³ is substituted then R³ is substituted with one or more groups selected from halogen, —CN, —OH, C₁-C₄alkyl, C₂-C₄alkenyl, C₂-C₄alkynyl, C₁-C₄alkoxy, C₁-C₄fluoroalkyl, C₁-C₄fluoroalkoxy, and substituted or unsubstituted C₁-C₄heteroalkyl.

In some embodiments, a compound of Formula A, or a pharmaceutically acceptable salt or solvate thereof, having the structure:

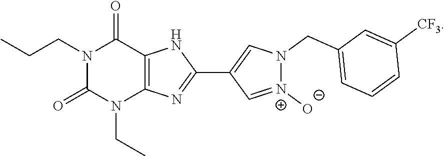 [SMILES: CCCn1c(=O)c2[nH]c(-c3cn(Cc4cccc(C(F)(F)F)c4)[n+]([O-])c3)nc2n(CC)c1=O]

Provided herein is a compound of Formula B, or a pharmaceutically acceptable salt or solvate thereof,

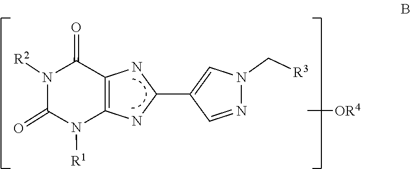

wherein
R¹ and R² are each independently selected from hydrogen and substituted or unsubstituted C₁-C₆ alkyl;
R³ is selected from substituted or unsubstituted phenyl and substituted or unsubstituted heteroaryl, wherein if R³ is substituted then R³ is substituted with one or more groups selected from halogen, —CN, —OH, C₁-C₄alkyl, C₂-C₄alkenyl, C₂-C₄alkynyl, C₁-C₄alkoxy, C₁-C₄fluoroalkyl, C₁-C₄fluoroalkoxy, and substituted or unsubstituted C₁-C₄heteroalkyl; and
R⁴ is hydrogen, or C₁-C₆ alkyl.

In some embodiments, a compound of Formula B, or a pharmaceutically acceptable salt or solvate thereof, selected from:

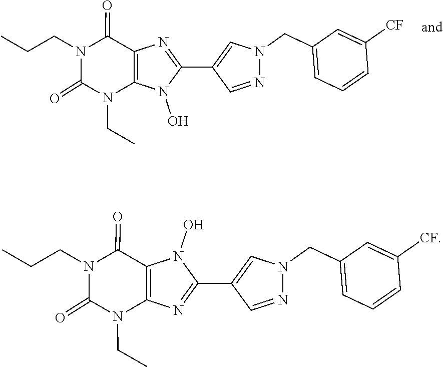

In some embodiments, provided herein is a compound selected from Table 1, or a pharmaceutically acceptable salt or solvate thereof.

TABLE 1

| Compound | Structure | Name |
|---|---|---|
| 1 | 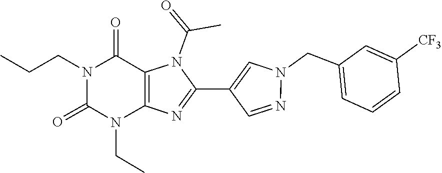 [SMILES: CCCn1c(=O)c2c(nc(-c3cnn(Cc4cccc(C(F)(F)F)c4)c3)n2C(C)=O)n(CC)c1=O] | 7-acetyl-3-ethyl-1-propyl-8- (1-(3- (trifluoromethyl)benzyl)- 1H-pyrazol-4-yl)-3,7- dihydro-1H-purine-2,6- dione |
| 2 | 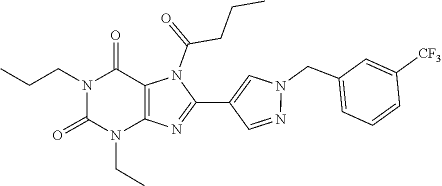 [SMILES: CCCC(=O)n1c(-c2cnn(Cc3cccc(C(F)(F)F)c3)c2)nc2c1c(=O)n(CCC)c(=O)n2CC] | 7-butyryl-3-ethyl-1-propyl- 8-(1-(3- (trifluoromethyl)benzyl)- 1H-pyrazol-4-yl)-3,7- dihydro-1H-purine-2,6- dione |
| 3 | 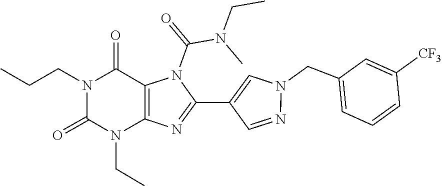 [SMILES: CCCn1c(=O)c2c(nc(-c3cnn(Cc4cccc(C(F)(F)F)c4)c3)n2C(=O)N(C)CC)n(CC)c1=O] | N,3-dicthyl-N-methyl-2,6- dioxo-1-propyl-8-(1-(3- (trifluoromethyl)benzyl)- 1H-pyrazol-4-yl)-1,2,3,6- tetrahydro-7H-purine-7- carboxamide |
| 4 | 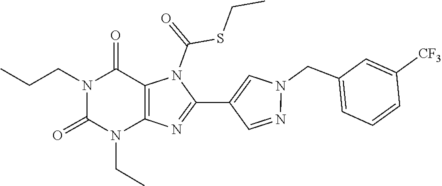 [SMILES: CCCn1c(=O)c2c(nc(-c3cnn(Cc4cccc(C(F)(F)F)c4)c3)n2C(=O)SCC)n(CC)c1=O] | S-ethyl 3-ethyl-2,6-dioxo-1- propyl-8-(1-(3- (trifluoromethyl)benzyl)- 1H-pyrazol-4-yl)-1,2,3,6- tetrahydro-7H-purine-7- carbothioate |
| 5 | 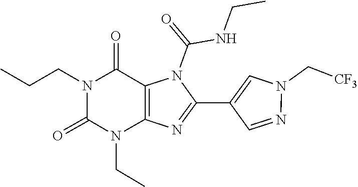 [SMILES: CCCn1c(=O)c2c(nc(-c3cnn(CC(F)(F)F)c3)n2C(=O)NCC)n(CC)c1=O] | N,3-diethyl-2,6-dioxo-1- propyl-8-(1-(2,2,2- trifluoroethyl)-1H-pyrazol- 4-yl)-1,2,3,6-tetrahydro-7H- purine-7-carboxamide |
| 6 | 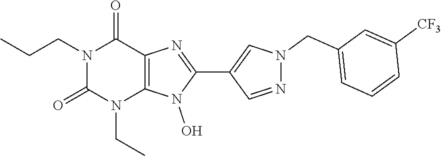 [SMILES: CCCn1c(=O)c2nc(-c3cnn(Cc4cccc(C(F)(F)F)c4)c3)n(O)c2n(CC)c1=O] | 3-ethyl-2,6-dioxo-1-propyl- 8-(1-(3- (trifluoromethyl)benzyl)- 1H-pyrazol-4-yl)-2,3,6,7- tetrahydro-1H-purine 9- oxide |
| 7 | 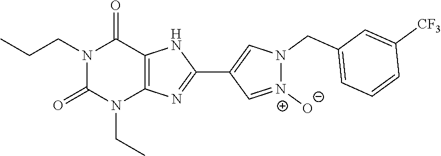 [SMILES: CCCn1c(=O)c2[nH]c(-c3cn(Cc4cccc(C(F)(F)F)c4)[n+]([O-])c3)nc2n(CC)c1=O] | 4-(3-ethyl-2,6-dioxo-1- propyl-2,3,6,7-tetrahydro- 1H-purin-8-yl)-1-(3- (trifluoromethyl)benzyl)- 1H-pyrazole 2-oxide |
| 8 | 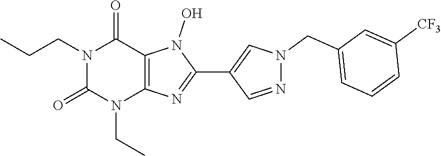 [SMILES: CCCn1c(=O)c2c(nc(-c3cnn(Cc4cccc(C(F)(F)F)c4)c3)n2O)n(CC)c1=O] | 3-ethyl-7-hydroxy-1-propyl- 8-(1-(3- (trifluoromethyl)benzyl)- 1H-pyrazol-4-yl)-3,7- dihydro-1H-purine-2,6- dione |
| 9 | 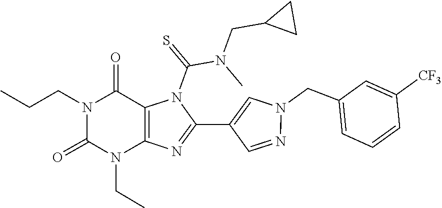 [SMILES: CCCn1c(=O)c2c(nc(-c3cnn(Cc4cccc(C(F)(F)F)c4)c3)n2C(=S)N(C)CC2CC2)n(CC)c1=O] | N-(cyclopropylmethyl)-3- ethyl-N-methyl-2,6-dioxo- 1-propyl-8-(1-(3- (trifluoromethyl)benzyl)- 1H-pyrazol-4-yl)-1,2,3,6- tetrahydro-7H-purine-7- carbothioamide |
| 10 | 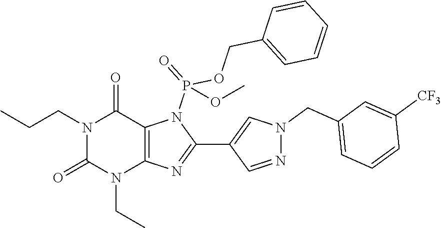 [SMILES: CCCn1c(=O)c2c(nc(-c3cnn(Cc4cccc(C(F)(F)F)c4)c3)n2P(=O)(OC)OCc2ccccc2)n(CC)c1=O] | benzyl methyl (3-ethyl-2,6- dioxo-1-propyl-8-(1-(3- (trifluoromethyl)benzyl)- 1H-pyrazol-4-yl)-1,2,3,6- tetrahydro-7H-purin-7- yl)phosphonate |
| 11 | 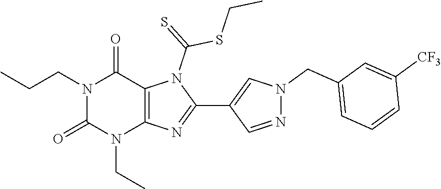 [SMILES: CCCn1c(=O)c2c(nc(-c3cnn(Cc4cccc(C(F)(F)F)c4)c3)n2C(=S)SCC)n(CC)c1=O] | ethyl 3-ethyl-2,6-dioxo-1- propyl-8-(1-(3- (trifluoromethyl)benzyl)- 1H-pyrazol-4-yl)-1,2,3,6- tetrahydro-7H-purine-7- carbodithioate |
| 12 | 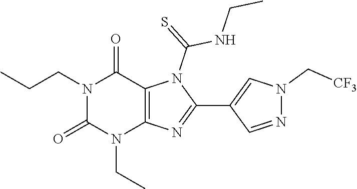 [SMILES: CCCn1c(=O)c2c(nc(-c3cnn(CC(F)(F)F)c3)n2C(=S)NCC)n(CC)c1=O] | N,3-diethyl-2,6-dioxo-1- propyl-8-(1-(2,2,2- trifluoroethyl)-1H-pyrazol- 4-yl)-1,2,3,6-tetrahydro-7H- purine-7-carbothioamide |
| 13 | 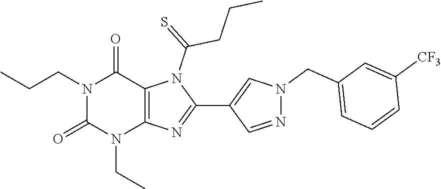 [SMILES: CCCC(=S)n1c(-c2cnn(Cc3cccc(C(F)(F)F)c3)c2)nc2c1c(=O)n(CCC)c(=O)n2CC] | 3-ethyl-1-propyl-7- (propylsulfinyl)-8-(1-(3- (trifluoromethyl)benzyl)- 1H-pyrazol-4-yl)-3,7- dihydro-1H-purine-2,6- dionc |
| 14 | 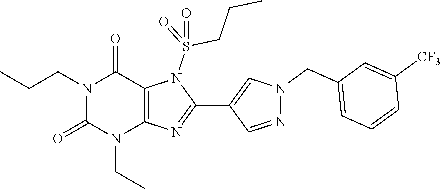 [SMILES: CCCn1c(=O)c2c(nc(-c3cnn(Cc4cccc(C(F)(F)F)c4)c3)n2S(=O)(=O)CCC)n(CC)c1=O] | 3-ethyl-1-propyl-7- (propylsulfonyl)-8-(1-(3- (trifluoromethyl)benzyl)- 1H-pyrazol-4-yl)-3,7- dihydro-1H-purine-2,6- dione |
| 15 | 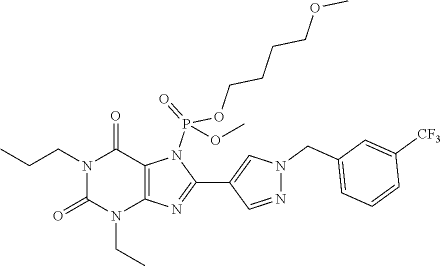 [SMILES: CCCn1c(=O)c2c(nc(-c3cnn(Cc4cccc(C(F)(F)F)c4)c3)n2P(=O)(OC)OCCCCOC)n(CC)c1=O] | 4-methoxybutyl methyl (3- ethyl-2,6-dioxo-1-propyl-8- (1-(3- (trifluoromethyl)benzyl)- 1H-pyrazol-4-y1)-1,2,3,6- tetrahydro-7H-purin-7- yl)phosphonate |
| 16 | 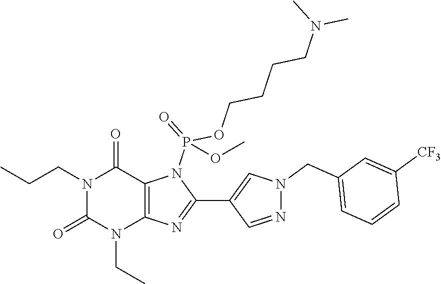 [SMILES: CCCn1c(=O)c2c(nc(-c3cnn(Cc4cccc(C(F)(F)F)c4)c3)n2P(=O)(OC)OCCCCN(C)C)n(CC)c1=O] | 4-(dimethylamino)butyl methyl (3-ethyl-2,6-dioxo- 1-propyl-8-(1-(3- (trifluoromethyl)benzyl)- 1H-pyrazol-4-yl)-1,2,3,6- tetrahydro-7H-purin-7- yl)phosphonate |
| 17 | 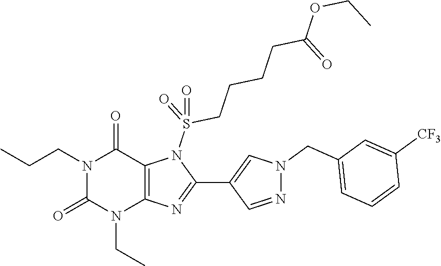 [SMILES: CCCn1c(=O)c2c(nc(-c3cnn(Cc4cccc(C(F)(F)F)c4)c3)n2S(=O)(=O)CCCCC(=O)OCC)n(CC)c1=O] | ethyl 5-((3-ethyl-2,6-dioxo- 1-propyl-8-(1-(3- (trifluoromethyl)benzyl)- 1H-pyrazol-4-yl)-1,2,3,6- tetrahydro-7H-purin-7- yl)sulfonyl)pentanoate |
| 18 | 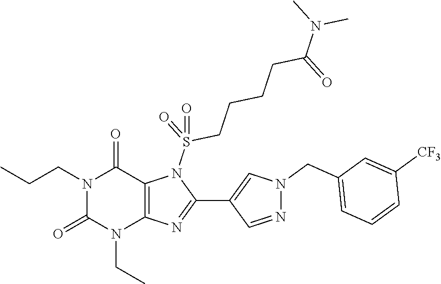 [SMILES: CCCn1c(=O)c2c(nc(-c3cnn(Cc4cccc(C(F)(F)F)c4)c3)n2S(=O)(=O)CCCCC(=O)N(C)C)n(CC)c1=O] | 5-((3-ethyl-2,6-dioxo-1- propyl-8-(1-(3- (trifluoromethyl)benzyl)- 1H-pyrazol-4-yl)-1,2,3,6- tetrahydro-7H-purin-7- yl)sulfonyl)-N,N- dimethylpentanamide |
| 19 | 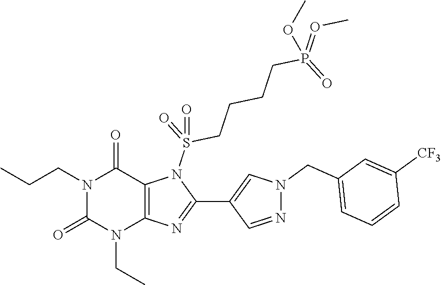 | dimethyl (4-((3-ethyl-2,6- dioxo-1-propyl-8-(1-(3- (trifluoromethyl)benzyl)- 1H-pyrazol-4-yl)-1,2,3,6- tetrahydro-7H-purin-7- yl)sulfonyl)butyl)phosphonate |
| 20 | 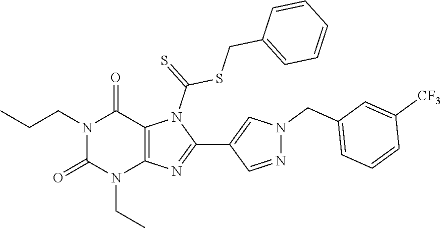 [SMILES: CCCn1c(=O)c2c(nc(-c3cnn(Cc4cccc(C(F)(F)F)c4)c3)n2C(=S)SCc2ccccc2)n(CC)c1=O] | benzyl 3-ethyl-2,6-dioxo-1- propyl-8-(1-(3- (trifluoromethyl)benzyl)- 1H-pyrazol-4-yl)-1,2,3,6- tetrahydro-7H-purine-7- carbodithioate |

### Treatment Methods and Uses

“Treatment” or “treating” is an approach for obtaining beneficial or desired results including clinical results. Beneficial or desired clinical results may include one or more of the following: a) inhibiting the disease or condition (e.g., decreasing one or more symptoms resulting from the disease or condition, and/or diminishing the extent of the disease or condition); b) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival.

“Prevention” or “preventing” means any treatment of a disease or condition that causes the clinical symptoms of the disease or condition not to develop. Compounds may, in some embodiments, be administered to a subject (including a human) who is at risk or has a family history of the disease or condition.

“Subject” refers to an animal, such as a mammal (including a human), that has been or will be the object of treatment, observation or experiment. The methods described herein may be useful in human therapy and/or veterinary applications. In some embodiments, the subject is a mammal. In one embodiment, the subject is a human.

The term “therapeutically effective amount” or “effective amount” of a compound described herein or a pharmaceutically acceptable salt, tautomer, stereoisomer, mixture of stereoisomers, prodrug, or deuterated analog thereof means an amount sufficient to effect treatment when administered to a subject, to provide a therapeutic benefit such as amelioration of symptoms or slowing of disease progression. For example, a therapeutically effective amount may be an amount sufficient to decrease a symptom of a disease or condition of cancer. The therapeutically effective amount may vary depending on the subject, and disease or condition being treated, the weight and age of the subject, the severity of the disease or condition, and the manner of administering, which can readily be determined by one or ordinary skill in the art.

The methods described herein may be applied to cell populations in vivo or ex vivo. “In vivo” means within a living individual, as within an animal or human. In this context, the methods described herein may be used therapeutically in an individual. “Ex vivo” means outside of a living individual. Examples of ex vivo cell populations include in vitro cell cultures and biological samples including fluid or tissue samples obtained from individuals. Such samples may be obtained by methods well known in the art. Exemplary biological fluid samples include blood, cerebrospinal fluid, urine, and saliva. In this context, the compounds and compositions described herein may be used for a variety of purposes, including therapeutic and experimental purposes. For example, the compounds and compositions described herein may be used ex vivo to determine the optimal schedule and/or dosing of administration of a compound of the present disclosure for a given indication, cell type, individual, and other parameters. Information gleaned from such use may be used for experimental purposes or in the clinic to set protocols for in vivo treatment. Other ex vivo uses for which the compounds and compositions described herein may be suited are described below or will become apparent to those skilled in the art. The selected compounds may be further characterized to examine the safety or tolerance dosage in human or non-human subjects. Such properties may be examined using commonly known methods to those skilled in the art.

In one aspect, the compounds, compositions, and/or formulations disclosed herein can be used to treat cancer. On endothelial cells, for example, adenosine can bind to the A_{2B} adenosine receptors, thereby stimulating angiogenesis. On T cells, A_{2B} adenosine receptor stimulation can lead to type I protein kinase A (PKA) isoform activation that can hamper T cell activation through inhibition of T-cell antigen receptor (TCR) proximal kinases Lck and Fyn. The pro-metastatic Fra-1 transcription factor can also induce A_{2B} adenosine receptor expression on cancer cells, and thus A_{2B} adenosine receptor antagonist can inhibit metastasis of Fra-1-expressing cells. A_{2B} adenosine receptor signaling activation can impair antigen presentation and can also inhibit signal transducer and activator of transcription 1 (STAT1) activation.

In addition, A_{2B}/cAMP/PKA potently dampens the immune response via inhibiting DCs function and stimulating immunosuppressive cells, such as myeloid-derived suppressor cells (MDSCs) and Tregs. A_{2B} activation impairs tumor antigen presentation on myeloid cells and DCs, decreases production of pro-inflammatory cytokines (TNF-α and IL-12) and increases immunosuppressive IL-10, resulting in a lower expression of CD86 and MHC class II and less efficient CD4+ T cell stimulation and antitumor responses. A_{2B} activation promotes the expansion of MDSCs, which potently suppresses antitumor T cell response and promotes angiogenesis. A_{2B} activation also stimulates Tregs differentiation to suppress T cell function. Besides, A_{2B} activation can contribute to the pro-angiogenic effects by increasing the production of vascular endothelial growth factor (VEGF) in endothelial cells. Taken together, A_{2B} plays an important role in tumor cell proliferation, angiogenesis, metastasis and immune suppression. The diversity of signaling and biological activities of A_{2B} adenosine receptor can render it an attractive cancer target to promote anti-tumor immunity and suppress tumor cell metastasis.

In another aspect, the compounds, compositions, and/or formulations disclosed herein can be used to treat fibrosis. A commonly ingested adenosine receptor antagonist, caffeine, can block the development of hepatic fibrosis, an effect that may explain the epidemiologic finding that coffee drinking, in a dose-dependent fashion, can reduce the likelihood of death from liver disease. A_{2B} adenosine receptors can also play a role in the pathogenesis of interstitial fibrosis. Adenosine, acting at A_{2B} adenosine receptors, can stimulate hepatic stellate cell-mediated fibrosis of the liver by increasing production of collagen I and III via two distinct mitogen-activated protein kinase (MAPK)-dependent pathways, extracellular signal-regulated kinase 1/2 (ERK1/2) and p38MAPK, respectively. Over-activation of A_{2B} adenosine receptors can be involved in liver, lung and heart fibrosis. Accordingly, A_{2B} adenosine receptors may be a good therapeutic target for fibrosis of the liver, lungs, heart, kidney, and/or skin. Applicant has found that Compound 1 reduces fibrosis in a MC38 tumor model, suggesting that Compound 1 could improve the tumor microenvironment for T cell function and infiltration and therapeutic antibody (such as anti-PD-1 antibody) penetration.

In another aspect, the compounds, compositions, and/or formulations disclosed herein can be used to treat diabetes and/or obesity. Insensitivity to insulin can exacerbate diabetes and/or obesity. Insulin sensitivity can be decreased by the interaction of adenosine with A_{2B} adenosine receptors. Thus, blocking the A_{2B} adenosine receptors of individuals with diabetes and/or obesity can benefit patients with these disorders.

In another aspect, the compounds, compositions, and/or formulations disclosed herein can be used to treat neurological disorders, such as dementias and Alzheimer's disease. Adenosine acting at A_{2B} adenosine receptors can over-stimulate cerebral interleukin 6 (IL-6), a cytokine associated with dementias and Alzheimer's disease. Inhibiting the binding of adenosine to A_{2B} adenosine receptors can therefore mitigate those neurological disorders that are produced by IL-6.

In another aspect, the compounds, compositions, and/or formulations disclosed herein can be used to treat type I hypersensitivity disorders, such as chronic obstructive pulmonary disease (COPD), asthma, hay fever, and atopic eczema. These type I hypersensitivity disorders can be stimulated by mast cells binding to A_{2B} adenosine receptors. Therefore, blocking A_{2B} adenosine receptors can provide a therapeutic benefit against such disorders.

In another aspect, blocking A_{2B} adenosine receptors may provide a therapeutic benefit against sickle cell disease. In another aspect, the compounds, compositions, and/or formulations disclosed herein can be used to treat irritable bowel disease (IBD) and/or colitis. Certain hypersensitivity disorders can be stimulated by mast cells binding to A_{2B} adenosine receptors. Therefore, blocking A_{2B} adenosine receptors can provide a therapeutic benefit against IBD and/or colitis.

### Combination Therapies

In one embodiment, the compounds disclosed herein may be used in combination with one or more additional therapeutic agent that are being used and/or developed to treat a condition described herein.

In some embodiments, a compound described herein, or a pharmaceutically acceptable salt thereof, is administered in combination with chemotherapy, radiation therapy, monoclonal antibodies, or combinations thereof. Chemotherapy includes the use of anti-cancer agents.

In some embodiments, a compound described herein (i.e. a A_{2B} adenosine receptor antagonist), or a pharmaceutically acceptable salt thereof, is administered in combination with an immune checkpoint inhibitor. In some embodiments, immune checkpoint inhibitors include, but are not limited to, anti-PD-1, anti-PD-L1, or anti-ligand 2 of programmed cell death protein 1 (PD-L2) agents/inhibitors. In some embodiments, immune checkpoint inhibitors include, but are not limited to anti-PD-1, anti-PD-L1, or anti-ligand 2 of programmed cell death protein 1 (PD-L2) antibodies.

As used herein, “PD-1” or “PD1” refers to the Programmed Death 1 (PD-1) receptor. Other names include programmed cell death protein 1 and CD279 (cluster of differentiation 279). PD-1 has two ligands, PD-L1 and PD-L2. In some embodiments, targeting PD-1 restores immune function in the tumor microenvironment.

As used herein, “PD-L1” or “PDL1” refers to the programmed death ligand 1 (PD-L1).

As used herein, “PD-L2” or “PDL2” refers to the programmed death ligand 2 (PD-L2).

In some embodiments, the anti-PD-1 or anti-PDL-1 agent is an antibody, a peptide, a small molecule or a nucleic acid.

In some embodiments, a compound described herein (i.e. a A_{2B} adenosine receptor antagonist), or a pharmaceutically acceptable salt thereof, is administered in combination with an anti-PD-1 or anti-PD-L1 agent. In some embodiments, the anti-PD-1 agent is an anti-PD-1 antibody. In some embodiments, the anti-PD-L1 agent is an anti-PD-L1 antibody.

In some embodiments, the anti PD-1 agent for use in combination with a compound described herein (i.e. a A_{2B} adenosine receptor antagonist), or a pharmaceutically acceptable salt thereof, is nivolumab, pembrolizumab, atezolizumab, durvalumab, pidilizumab, avelumab, TSR-042, PDR-001, tislelizumab (BGB-A317), cemiplimab (REGN2810), LY-3300054, JNJ-63723283, MGA012, BI-754091, IBI-308, camrelizumab (HR-301210), BCD-100, JS-001, CX-072, BGB-A333, AMP-514 (MEDI-0680), AGEN-2034, CSIOOI, Sym-021, SHR-1316, PF-06801591, LZMO9, KN-035, AB122, genolimzumab (CBT-501), FAZ-053, CK-301, AK 104, or GLS-010, BGB-108, SHR-1210, PDR-001, PF-06801591, STI-1110, mDX-400, Spartalizumah (PDR001), Camrelizumah (SHR1210), Sintilimab (IBI308), Tislelizumab (BGB-A317), Toripalimab (JS 001), Dostarlimab (TSR-042, WBP-285), INCMGA00012 (MGA012), AMP-224, or AMP-514 (MEDI0680).

In some embodiments, the anti PD-1 agent is an anti PD-1 antibody.

“Anti-PD-1 antibody” refers to an antibody directed towards programmed death protein 1 (PD1). In some embodiments, an anti-PD-1 antibody binds an epitope of PD-1 which blocks the binding of PD-1 to any one or more of its putative ligands. In some embodiments, an anti-PD1 antibody binds an epitope of a PD-1 protein which blocks the binding of PD-1 to PD-L1 and/or PD-L2.

Exemplary anti-PD-1 antibodies include but are not limited to: nivolumab/MDX-1106/BMS-9300/ONO1152, a fully human lgG4 anti-PD-1 monoclonal antibody; pidilizumab (MDV9300/CT-011), a humanized IgG1 monoclonal antibody; pembrolizumab (MK-3475/pembrolizumab/lambrolizumab), a humanized monoclonal IgG4 antibody; durvalumab (MEDI-4736) and atezolizumab.

In some embodiments, the anti-PD-1 antibody is nivolumab (OPDIVO®, Bristol-Myers Squibb), pembrolizumab (KEYTRUDA®, Merck), cemiplimab (Libtayo), labrolizumab (Merck), or BGB-A317.

In some embodiments, the anti-PD1 antibody is an antibody set forth in U.S. Pat. Nos. 7,029,674, 7,488,802, 7,521,051, 8,008,449, 8,354,509, 8,617,546, 8,709,417, or WO2014/179664.-

The terms “antibody” and “antibodies” as used herein is inclusive of all types of immunoglobulins, including IgG, IgM, IgA, IgD, and IgE, or fragments thereof, that may be appropriate for the medical uses disclosed herein. The antibodies may be monoclonal or polyclonal and may be of any species of origin, including, for example, mouse, rat, rabbit, horse, or human. Antibody fragments that retain specific binding to the protein or epitope, for example, PD-L1 or PD-1, bound by the antibody used in the present disclosure are included within the scope of the term “antibody.” The antibodies may be chimeric or humanized, particularly when they are used for therapeutic purposes. Antibodies and antibody fragments may be obtained or prepared using various methods.

In some embodiments, the anti PD-1 agent for use in combination with a compound described herein (i.e. a A_{2B} adenosine receptor antagonist), or a pharmaceutically acceptable salt thereof, is atezolizumab, avelumab, AMP-224, MEDI-0680, RG-7446, GX-P2, durvalumab, KY-1003, KD-033, MSB-0010718C, TSR-042, ALN-PDL, STI-A1014, CX—072, BMS-936559, KN035, CK-301 (Checkpoint Therapeutics), AUNP12, CA-170 (Aurigene/Curis), MEDI4736, MSB0010718C, MDX 1105-01, and BMS-986189.

In some embodiments, the anti PD-L1 agent is an anti PD-L1 antibody.

“Anti-PD-L1 antibody” refers to an antibody directed towards programmed death ligand 1 (PD-L1).

Anti-PD-L1 antibodies for use in combination with a compound described herein (i.e. a A_{2B} adenosine receptor antagonist), or a pharmaceutically acceptable salt thereof, include: avelumab; BMS-936559, a fully human IgG4 antibody; atezolizumab (MPDL3280A/RG-7446), a human monoclonal antibody; MEDI4736; MSB0010718C, and MDX 1105-01.

In some embodiments, the anti-PD-L1 antibody is avelumab (Bavencio®, Merck KGA/Pfizer), durvalumab (AstraZeneca) and atezolizumab (TECENTRIQ®, Roche).

Additional exemplary antibodies include, but are not limited to, the antibodies set forth in U.S. Pat. Nos. 8,217,149, 8,383,796, 8,552,154 and 8,617,546.

Peptide anti-PD-1/PD-L1 agents include AUNP12 (a 29-mer peptide by Aurigene and Laboratoires Pierre Fabre), CA-170 (Aurigene/Curis), BMS-986189 (a macrocyclic peptide by BMS).

Small molecule anti-PD-1/PD-L1 agents include those described in WO/2020/086556, WO/2020/014643, WO/2019/204609, WO/2019/160882, WO/2018/195321, WO2018026971, US20180044329, US20180044305, US20180044304, US20180044303, US20180044350, US20180057455, US20180057486, US20180045142, WO20180044963, WO2018044783, WO2018009505, WO20180044329, WO2017066227, WO2017087777, US20170145025, WO2017079669, WO2017070089, US2017107216, WO2017222976, US20170262253, WO2017205464, US20170320875, WO2017192961, WO2017112730, US20170174679, WO2017106634, WO2017202744, WO2017202275, WO2017202273, WO2017202274, WO2017202276, WO2017180769, WO2017118762, WO2016041511, WO2016039749, WO2016142835, WO2016142852, WO2016142886, WO2016142894, and WO2016142833. In some embodiments, the small molecule anti-PD-1/PD-L1 agent is GS-4224. In some embodiments, GS-4224 is administered at about 400 mg to about 1000 mg. In some embodiments, immune checkpoint inhibitors include, and are not limited to, anti-T cell immunoglobulin and anti-immunoreceptor tyrosine-based inhibition motif (ITIM) domain (anti-TIGIT) agents such as BMS-986207, which is an anti-TIGIT monoclonal antibody. In some embodiments, immune checkpoint inhibitors include, and are not limited to, anti-lymphocyte activation gene-3 (anti-LAG3) agents such as relatlimab (BMS). In some embodiments, immune checkpoint inhibitors include, and are not limited to, anti-vascular endothelial growth factor (anti-VEGF) agents such as ranibizumab (Lucentis®) and bevacizumab (Avastin®).

### Kits

Provided herein are also kits that include a compound of the disclosure, or a pharmaceutically acceptable salt, tautomer, stereoisomer, mixture of stereoisomers, prodrug, or deuterated analog thereof, and suitable packaging. In one embodiment, a kit further includes instructions for use. In one aspect, a kit includes a compound of the disclosure, or a pharmaceutically acceptable salt, tautomer, stereoisomer, mixture of stereoisomers, prodrug, or deuterated analog thereof, and a label and/or instructions for use of the compounds in the treatment of the indications, including the diseases or conditions, described herein.

Provided herein are also articles of manufacture that include a compound described herein or a pharmaceutically acceptable salt, tautomer, stereoisomer, mixture of stereoisomers, prodrug, or deuterated analog thereof in a suitable container. The container may be a vial, jar, ampoule, preloaded syringe, and intravenous bag.

### Pharmaceutical Compositions and Modes of Administration

Compounds provided herein are usually administered in the form of pharmaceutical compositions. Thus, provided herein are also pharmaceutical compositions that contain one or more of the compounds described herein or a pharmaceutically acceptable salt, tautomer, stereoisomer, mixture of stereoisomers, prodrug, or deuterated analog thereof and one or more pharmaceutically acceptable vehicles selected from carriers, adjuvants and excipients. Suitable pharmaceutically acceptable vehicles may include, for example, inert solid diluents and fillers, diluents, including sterile aqueous solution and various organic solvents, permeation enhancers, solubilizers and adjuvants. Such compositions are prepared in a manner well known in the pharmaceutical art. See, e.g., Remington's Pharmaceutical Sciences. Mace Publishing Co., Philadelphia. Pa. 17th Ed. (1985); and Modern Pharmaceutics. Marcel Dekker, Inc. 3rd Ed. (G. S. Banker & C. T. Rhodes, Eds.).

The pharmaceutical compositions may be administered in either single or multiple doses. The pharmaceutical composition may be administered by various methods including, for example, rectal, buccal, intranasal and transdermal routes. In certain embodiments, the pharmaceutical composition may be administered by intra-arterial injection, intravenously, intraperitoneally, parenterally, intramuscularly, subcutaneously, orally, topically, or as an inhalant.

One mode for administration is parenteral, for example, by injection. The forms in which the pharmaceutical compositions described herein may be incorporated for administration by injection include, for example, aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Oral administration may be another route for administration of the compounds described herein. Administration may be via, for example, capsule or enteric coated tablets. In making the pharmaceutical compositions that include at least one compound described herein or a pharmaceutically acceptable salt, tautomer, stereoisomer, mixture of stereoisomers, prodrug, or deuterated analog thereof, the active ingredient is usually diluted by an excipient and/or enclosed within such a carrier that can be in the form of a capsule, sachet, paper or other container. When the excipient serves as a diluent, it can be in the form of a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include lubricating agents such as talc, magnesium stearate, and mineral oil; wetting agents; emulsifying and suspending agents; preserving agents such as methyl and propylhydroxy-benzoates; sweetening agents; and flavoring agents.

The compositions that include at least one compound described herein or a pharmaceutically acceptable salt, tautomer, stereoisomer, mixture of stereoisomers, prodrug, or deuterated analog thereof can be formulated so as to provide quick, sustained or delayed release of the active ingredient after administration to the subject by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods disclosed herein employ transdermal delivery devices (“patches”). Such transdermal patches may be used to provide continuous or discontinuous infusion of the compounds described herein in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

For preparing solid compositions such as tablets, the principal active ingredient may be mixed with a pharmaceutical excipient to form a solid preformulation composition containing a homogeneous mixture of a compound described herein or a pharmaceutically acceptable salt, tautomer, stereoisomer, mixture of stereoisomers, prodrug, or deuterated analog thereof. When referring to these preformulation compositions as homogeneous, the active ingredient may be dispersed evenly throughout the composition so that the composition may be readily subdivided into equally effective unit dosage forms such as tablets, pills and capsules.

The tablets or pills of the compounds described herein may be coated or otherwise compounded to provide a dosage form affording the advantage of prolonged action, or to protect from the acid conditions of the stomach. For example, the tablet or pill can include an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Compositions for inhalation or insufflation may include solutions and suspensions in pharmaceutically acceptable, aqueous or organic solvents, or mixtures thereof, and powders. The liquid or solid compositions may contain suitable pharmaceutically acceptable excipients as described herein. In some embodiments, the compositions are administered by the oral or nasal respiratory route for local or systemic effect. In other embodiments, compositions in pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, preferably orally or nasally, from devices that deliver the formulation in an appropriate manner.

### Dosing

The specific dose level of a compound of the present application for any particular subject will depend upon a variety of factors including the activity of the specific compound employed, the age, body weight, general health, sex, diet, time of administration, route of administration, and rate of excretion, drug combination and the severity of the particular disease in the subject undergoing therapy. For example, a dosage may be expressed as a number of milligrams of a compound described herein per kilogram of the subject's body weight (mg/kg). Dosages of between about 0.1 and 150 mg/kg may be appropriate. In some embodiments, about 0.1 and 100 mg/kg may be appropriate. In other embodiments a dosage of between 0.5 and 60 mg/kg may be appropriate. Normalizing according to the subject's body weight is particularly useful when adjusting dosages between subjects of widely disparate size, such as occurs when using the drug in both children and adult humans or when converting an effective dosage in a non-human subject such as dog to a dosage suitable for a human subject.

The daily dosage may also be described as a total amount of a compound described herein administered per dose or per day. Daily dosage of a compound of Formula I may be between about 1 mg and 4,000 mg, between about 2,000 to 4,000 mg/day, between about 1 to 2,000 mg/day, between about 1 to 1,000 mg/day, between about 10 to 500 mg/day, between about 20 to 500 mg/day, between about 50 to 300 mg/day, between about 75 to 200 mg/day, or between about 15 to 150 mg/day.

When administered orally, the total daily dosage for a human subject may be between 1 mg and 1,000 mg, between about 1,000-2,000 mg/day, between about 10-500 mg/day, between about 50-300 mg/day, between about 75-200 mg/day, or between about 100-150 mg/day.

The compounds of the present application or the compositions thereof may be administered once, twice, three, or four times daily, using any suitable mode described above. Also, administration or treatment with the compounds may be continued for a number of days; for example, commonly treatment would continue for at least 7 days, 14 days, or 28 days, for one cycle of treatment. Treatment cycles are well known in cancer chemotherapy, and arc frequently alternated with resting periods of about 1 to 28 days, commonly about 7 days or about 14 days, between cycles. The treatment cycles, in other embodiments, may also be continuous.

In a particular embodiment, the method comprises administering to the subject an initial daily dose of about 1 to 800 mg of a compound described herein and increasing the dose by increments until clinical efficacy is achieved. Increments of about 5, 10, 25, 50, or 100 mg can be used to increase the dose. The dosage can be increased daily, every other day, twice per week, or once per week.

### Synthesis of the Compounds

The compounds may be prepared using the methods disclosed herein and routine modifications thereof, which will be apparent given the disclosure herein and methods well known in the art.

Conventional and well-known synthetic methods may be used in addition to the teachings herein. The synthesis of typical compounds described herein may be accomplished as described in the following examples. If available, reagents may be purchased commercially, e.g., from Sigma Aldrich or other chemical suppliers.

### General Synthesis

Typical embodiments of compounds described herein may be synthesized using the general reaction schemes described below. It will be apparent given the description herein that the general schemes may be altered by substitution of the starting materials with other materials having similar structures to result in products that are correspondingly different. Descriptions of syntheses follow to provide numerous examples of how the starting materials may vary to provide corresponding products. Given a desired product for which the substituent groups are defined, the necessary starting materials generally may be determined by inspection. Starting materials are typically obtained from commercial sources or synthesized using published methods. For synthesizing compounds which are embodiments described in the present disclosure, inspection of the structure of the compound to be synthesized will provide the identity of each substituent group. The identity of the final product will generally render apparent the identity of the necessary starting materials by a simple process of inspection, given the examples herein. In general, compounds described herein are typically stable and isolatable at room temperature and pressure.

Starting with compounds A-1, which are described in U.S. Pat. No. 6,825,349, a reaction with a suitable reactant A-2 provides compounds of Formula I as shown in Scheme 1. LG is a suitable leaving group such as chloro, bromo, triflate, or any other suitable leaving group known to one of skill in the art.

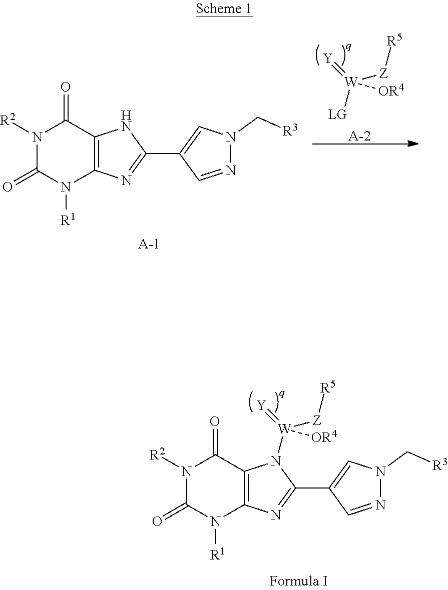

wherein R¹, R², R³, R⁴, R⁵, W, Y, Z, and q are as defined herein.

Similarly, oxidation of compounds A-1 can provide compounds of Formula A and Formula B.

### EXAMPLES

The following examples are included to demonstrate specific embodiments of the disclosure. It should be appreciated by those of skill in the art that the techniques disclosed in the examples which follow represent techniques to function well in the practice of the disclosure, and thus can be considered to constitute specific modes for its practice. However, those of skill in the art should, in light of the present disclosure, appreciate that many changes can be made in the specific embodiments which are disclosed and still obtain a like or similar result without departing from the spirit and scope of the disclosure.

General methods Preparative HPLC was performed on a Shimadzu LC-20AP. The preparative system was fitted with a Phenomenex Luna C18 150*25 mm*10 μm reverse-phase column at 22 degree Celsius. The mobile phase consisted of a mixture of solvent 0.1% TFA in water and MeCN (TFA condition); 0.225% FA in water and MeCN (FA condition). A constant gradient from 100% aqueous/0% organic to 0% aqueous/100% organic mobile phase over the course of 25 minutes was utilized. The flow rate was constant at 25 mL/min.

### Example 1—Synthesis of 7-acetyl-3-ethyl-1-propyl-8-(1-(3-(trifluoromethyl)benzyl)-1H-pyrazol-4-yl)-3,7-dihydro-1H-purine-2,6-dione (Compound 1)

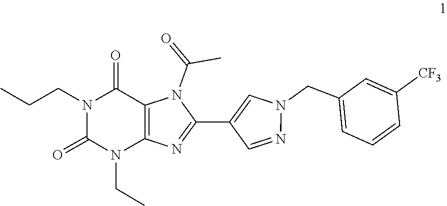 [SMILES: CCCn1c(=O)c2c(nc(-c3cnn(Cc4cccc(C(F)(F)F)c4)c3)n2C(C)=O)n(CC)c1=O]

### Preparation of 7-acetyl-3-ethyl-1-propyl-8-(1-(3-(trifluoromethyl)benzyl)-1H-pyrazol-4-yl)-3,7-dihydro-1H-purine-2,6-dione

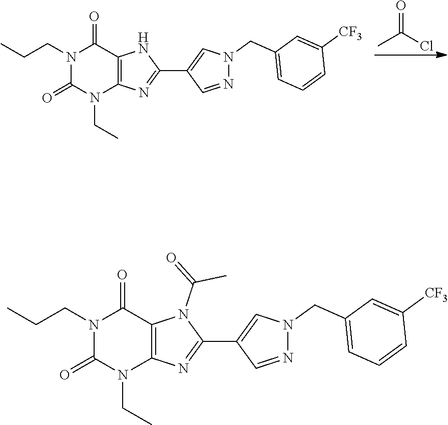

To a solution of 3-ethyl-1-propyl-8-(1-(3-(trifluoromethyl)benzyl)-1H-pyrazol-4-yl)-3,7-dihydro-1H-purine-2,6-dione (3 g, 6.72 mmol, 1 eq) in dry DMF (30 mL) was added triethylamine (3 mL) under nitrogen atmosphere. After stirred at 0° C. for 5 min, a solution of acetyl chloride (791.26 mg, 10.08 mmol, 719.33 uL, 1.5 eq) in dry DMF (10 mL) was added dropwise. The reaction mixture was stirred for additional 5 min at room temperature after addition, and monitored by TLC. After completion, the mixture was quenched with ice-cold water and extracted with ethyl acetate (3×50 mL). The organic layer was dried over anhydrous sodium sulfate and concentrated under reduced pressure. The residue was purified by silica gel chromatograph and washed with ethyl acetate/petroleum ether (1:6). The desired Compound 1 was obtained as a white solid (1 g, 2.05 mmol, 30.46% yield).

¹H NMR (400 MHz, CD₃OD) δ 8.56 (s, 1H), 7.99 (s, 1H), 7.71-7.68 (m, 2H), 7.64-7.61 (m, 2H), 5.52 (s, 2H), 4.10-4.05 (m, 2H), 3.87-3.83 (m, 2H), 2.79 (s, 3H), 1.63-1.53 (m, 2H), 1.26 (t, J=7.4 Hz, 3H), 0.87 (t, J=7.4 Hz, 3H). LCMS (ES+): m/z 489.1 [M+H]+.

### Example 2—Synthesis of 7-butyryl-3-ethyl-1-propyl-8-(1-(3-(trifluoromethyl)benzyl)-1H-pyrazol-4-yl)-3,7-dihydro-1H-purine-2,6-dione (Compound 2)

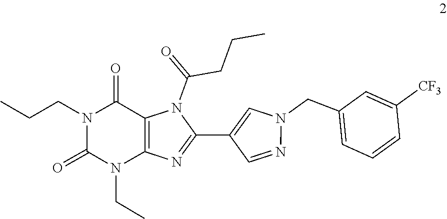 [SMILES: CCCC(=O)n1c(-c2cnn(Cc3cccc(C(F)(F)F)c3)c2)nc2c1c(=O)n(CCC)c(=O)n2CC]

### Preparation of 7-butyryl-3-ethyl-1-propyl-8-(1-(3-(trifluoromethyl)benzyl)-1H-pyrazol-4-yl)-3,7-dihydro-1H-purine-2,6-dione

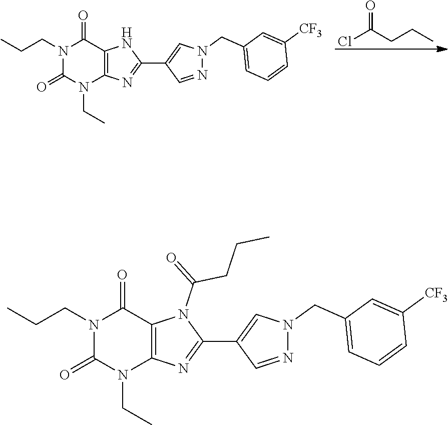

To a solution of 3-ethyl-1-propyl-8-(1-(3-(trifluoromethyl)benzyl)-1H-pyrazol-4-yl)-3,7-dihydro-1H-purine-2,6-dione (3 g, 6.72 mmol, 1 eq) in dry DMF (30 mL) was added triethylamine (3 mL) under nitrogen atmosphere. After stirred at 0° C. for 5 min, a solution of butyryl chloride (1.07 g, 10.08 mmol, 1.05 mL, 1.5 eq) in dry DMF (10 mL) was added dropwise. The reaction mixture was stirred for additional 5 min at room temperature after addition, and monitored by TLC. After completion, the mixture was quenched with ice-cold water and extracted with ethyl acetate (3×50 mL). The organic layer was dried over anhydrous sodium sulfate and concentrated under reduced pressure. The residue was purified by silica gel chromatograph and washed with ethyl acetate/petroleum ether (1:6). The desired Compound 2 was obtained as a white solid (1.1 g, 2.25 mmol, 33.51% yield).

¹H NMR (400 MHz, CD₃OD): δ 8.52 (s, 1H), 7.95 (s, 1H), 7.70-7.67 (m, 2H), 7.63-7.60 (m, 2H), 5.56 (s, 2H), 4.07 (d, J=7.2 Hz, 2H), 3.87-3.83 (m, 2H), 3.15 (t, J=7.2 Hz, 2H), 1.66-1.56 (m, 4H), 1.26 (t, J=7.2 Hz, 3H), 0.89-0.84 (m, 6H). LCMS (ESI+): m/z 517.1 [M+H]+.

### Example 3—Synthesis of N,3-diethyl-N-methyl-2,6-dioxo-1-propyl-8-(1-(3-(trifluoromethyl)benzyl)-1H-pyrazol-4-yl)-1,2,3,6-tetrahydro-7H-purine-7-carboxamide (Compound 3) and N,3-diethyl-N-methyl-2,6-dioxo-1-propyl-8-(1-(3-(trifluoromethyl)benzyl)-1H-pyrazol-4-yl)-1,2,3,6-tetrahydro-9H-purine-9-carboxamide (Compound 3A)

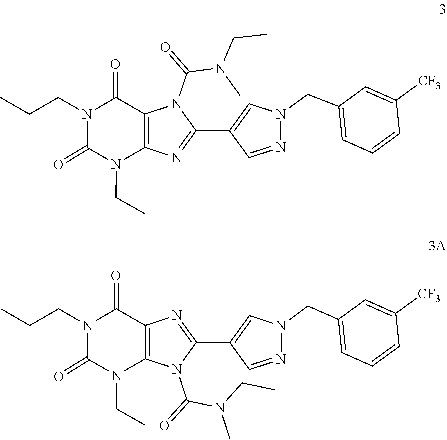 [SMILES: CCCn1c(=O)c2c(nc(-c3cnn(Cc4cccc(C(F)(F)F)c4)c3)n2C(=O)N(C)CC)n(CC)c1=O ; CCCn1c(=O)c2nc(-c3cnn(Cc4cccc(C(F)(F)F)c4)c3)n(C(=O)N(C)CC)c2n(CC)c1=O]

### Preparation of N,3-diethyl-N-methyl-2,6-dioxo-1-propyl-8-(1-(3-(trifluoromethyl)benzyl)-1H-pyrazol-4-yl)-1,2,3,6-tetrahydro-7H-purine-7-carboxamide and N,3-diethyl-N-methyl-2,6-dioxo-1-propyl-8-(1-(3-(trifluoromethyl)benzyl)-1H-pyrazol-4-yl)-1,2,3,6-tetrahydro-9H-purine-9-carboxamide

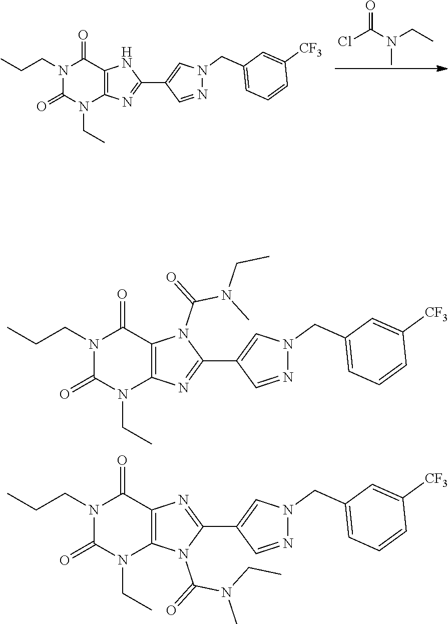

To a solution of 3-ethyl-1-propyl-8-(1-(3-(trifluoromethyl)benzyl)-1H-pyrazol-4-yl)-3,7-dihydro-1H-purine-2,6-dione (3 g, 6.72 mmol, 1 eq) in dry DMF (20 mL) was added NaH (806.32 mg, 33.60 mmol, 5 eq) under nitrogen atmosphere. After stirred at 0° C. for 5 min, a solution of N-ethyl-N-methyl-carbamoyl chloride (4.08 g, 33.60 mmol, 1.05 mL, 5 eq) in dry THF (20 mL) was added dropwise. The reaction mixture was stirred at 80° C. for 10 h, monitored by TLC. After completion, the mixture was quenched with ice-cold water and extracted with ethyl acetate (3×50 mL). The organic layer was dried over anhydrous sodium sulfate and concentrated under reduced pressure. The residue was purified by silica gel chromatograph and washed with ethyl acetate/petroleum ether (1:6). The desired Compound 3 was obtained as a white solid (1.5 g, 3.07 mmol, 45.70% yield).

HPLC showed two peaks with retention times as 36.16 and 36.86 minute and peak ration of 1:3. The HPLC condition is 10-80_AB_60 min: HPLC (The gradient was 10-80% B in 55.00 min with a hold at 80% B for 5.00 min, 80-10% B in 0.01 min, and then held at 10% for 1.0 min(2.0 m/min flow rate). Mobile phase A was 0.04% trifluoroacetic acid in water, mobile phase B was 0.02% trifluoroacetic acid in acetonitrile. The column used for chromatography was a 4.6*150 mm Gemini-NX C18 column (5 μm particles). Detection methods are diode array (DAD).

¹H NMR (400 MHz, CDCl₃): δ 8.66 (s, 0.3H), 8.47-8.45 (m, 0.7H), 8.08 (s, 0.3H), 7.86 (s, 0.7H), 7.70-7.68 (m, 2H), 7.64-7.60 (m, 2H), 5.55 (s, 2H), 4.06-4.03 (m, 2H), 3.85-3.80 (m, 2H), 3.57-3.47 (m, 1.5H), 3.33 (s, 1H), 3.08-3.02 (m, 0.5H), 2.70 (s, 1H), 1.57-1.54 (m, 2H), 1.28-1.24 (m, 3H), 1.20-1.16 (m, 2H), 0.87-0.84 (m, 4H). LCMS (ESI+): m/z 532.2 [M+H]+.

### Example 4—Synthesis of S-ethyl 3-ethyl-2,6-dioxo-1-propyl-8-(1-(3-(trifluoromethyl)benzyl)-1H-pyrazol-4-yl)-1,2,3,6-tetrahydro-7H-purine-7-carbothioate (Compound 4)

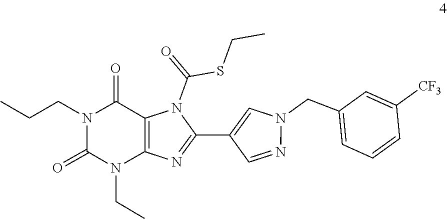 [SMILES: CCCn1c(=O)c2c(nc(-c3cnn(Cc4cccc(C(F)(F)F)c4)c3)n2C(=O)SCC)n(CC)c1=O]

### Preparation of S-ethyl 3-ethyl-2,6-dioxo-1-propyl-8-(1-(3-(trifluoromethyl)benzyl)-1H-pyrazol-4-yl)-1,2,3,6-tetrahydro-7H-purine-7-carbothioate

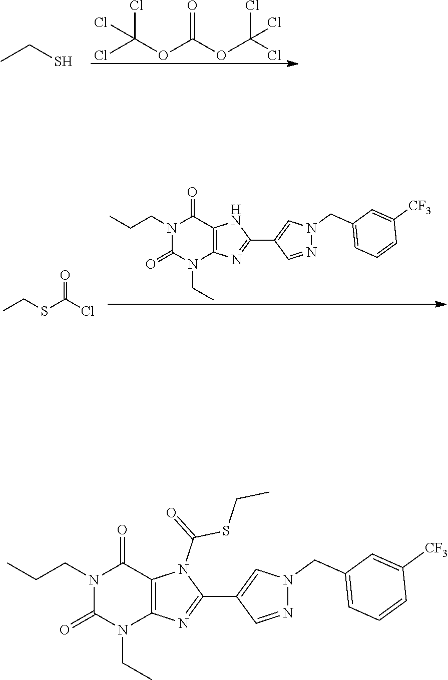

Bis(trichloromethyl) carbonate (23.88 g, 80.47 mmol, 1 eq) was added into a mixture of ethanethiol (5 g, 80.47 mmol, 5.95 mL, 1 eq) and triethylamine (8.14 g, 80.47 mmol, 11.20 mL, 1 eq) in THF (50 mL) at 0° C. and stirred at 25° C. for 1 h. Then the above solution was added into a solution of 3-ethyl-1-propyl-8-(1-(3-(trifluoromethyl)benzyl)-1H-pyrazol-4-yl)-3,7-dihydro-1H-purine-2,6-dione (3 g, 6.72 mmol, 1 eq) and pyridine (1.06 g, 13.44 mmol, 1.08 mL, 2 eq) in THF (30 mL) at 0° C. and stirred at 10° C. for 10 h, monitored by TLC. After completion, the mixture was quenched with ice-cold water and extracted with ethyl acetate (3×50 mL). The organic layer was dried over anhydrous sodium sulfate and concentrated under reduced pressure. The residue was purified by silica gel chromatograph and washed with ethyl acetate/petroleum ether (1:6). The desired Compound 4 was obtained as a white solid (200 mg, 374.14 μmol, 5.57% yield).

¹H NMR (400 MHz, CDCl₃): δ 8.53 (s, 1H), 7.93 (s, 1H), 7.70-7.68 (m, 2H), 7.62-7.60 (m, 2H), 5.56 (s, 2H), 4.05 (q, J=7.2 Hz, 2H), 3.83-3.79 (m, 2H), 3.19-3.13 (m, 2H), 1.58-1.53 (m, 2H), 1.32-1.25 (m, 6H), 0.88-0.84 (m, 3H). LCMS (ESI+): m/z 535.1 [M+H]+.

### BIOLOGICAL ASSAYS

### Example 1—Pharmcokinetic Properties

Pharmacokinetic studies are carried out in Sprague Dawley rats. Exemplary compounds are administered orally by gavage to groups of three rats using a single oral dose of 5 mg/kg. Each oral dose is prepared as a suspension in 0.5% methylcellulose in water. Blood samples are obtained serially from each rat at 0, 15, 30 min, and then 1, 2, 4, 8, and 24 hrs post dose. Concentrations of an administered compound and the corresponding metabolite (Compound 1) in rat plasma are determined by a HPLC tandem mass spectrometric (LC/MS/MS) method.

### Example 2—Anti-Tumor Activity

Balb/C inbred female mice, aged 8-9 weeks, are purchased. On the day of inoculation (Day 0), CT26 cells (murine colorectal carcinoma cell line) are harvested, washed and counted. Cells are re-suspended as single cell solution in PBS at a concentration of 5×10⁶ cells/mL at the final step. Immediately, five hundred thousand (5×10⁵) of CT26 cells suspended in 0.1 mL PBS are injected in the right flank of Balb/C mice subcutaneously using 27G needles. When palpable, tumors are measurable by a caliper, tumor volume (mm³) is calculated by length×width×height×0.5236. Mice with the tumor size approximate to 100 mm³ are randomly assigned into one of four groups (n=10). Each group receives vehicle (BID), or A_{2B} antagonist compound at 3 mg/kg (BID), Mouse anti-PD-1 (RMP1-14) at 5 mg/kg (Q2D), or Compound (3 mg/kg)+RMP1-14 (5 mg/kg) intraperitoneally for 16 days. Tumor size and body weight are determined every 2-3 days.

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

1. A compound of Formula I, or a pharmaceutically acceptable salt or solvate thereof, 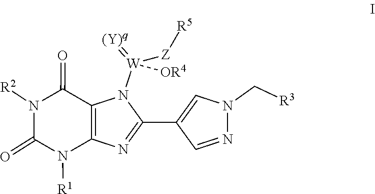
wherein
the dotted line - - indicates that the referenced group may be present or absent;
when W is C and Y is O, then q is 1, Z is absent, —S—, or —N—(R⁴)—, and OR⁴ is absent;
when W is C and Y is S, then q is 1, Z is absent, —O—, —S—, or —N—(R⁴)—, and OR⁴ is absent;
when W is C and Y is —N—(R⁴), then q is 1, Z is —CH₂— or —N—(R⁴)—, and OR⁴ is absent;
when W is P and Y is O, then q is 1, Z is —O— or —N—(R⁴)—, and OR⁴ is present;
when W is S and Y is O or —N(R⁴), then q is 1 or 2, Z is —CH₂— or —N—(R⁴)—, and OR⁴ is absent;
R¹ and R² are each independently selected from hydrogen and substituted or unsubstituted C₁-C₆ alkyl;
R³ is selected from substituted or unsubstituted phenyl and substituted or unsubstituted heteroaryl, wherein if R³ is substituted then R³ is substituted with one or more groups selected from halogen, —CN, —OH, C₁-C₄alkyl, C₂-C₄alkenyl, C₂-C₄alkynyl, C₁-C₄alkoxy, C₁-C₄fluoroalkyl, C₁-C₄fluoroalkoxy, and substituted or unsubstituted C₁-C₄heteroalkyl;
each R⁴ is independently hydrogen or C₁-C₆ alkyl;
R⁵ is hydrogen, R⁷, —C(═O)R⁷, —C(═O)—OR⁷, —C(═O)N(R⁷)(R⁸), —C(═O)—SR⁷ or —P(═O)(OR⁹)₂;
R⁷ is substituted or unsubstituted C₁-C₆ alkyl, substituted or unsubstituted heteroalkyl, substituted or unsubstituted C₃-C₁₀cycloalkyl, substituted or unsubstituted C₂-C₁₀heterocycloalkyl, substituted or unsubstituted phenyl, substituted or unsubstituted heteroaryl, -alkyl-(substituted or unsubstituted phenyl), -alkyl-(substituted or unsubstituted heteroaryl), -alkyl-(substituted or unsubstituted cycloalkyl), -alkyl-(substituted or unsubstituted heterocycloalkyl), —(C(R¹⁰)₂O)ₘ—R¹¹, —(CH₂CH₂O)ₙ—R¹¹, or —(C(R¹⁰)₂)ₚ—OR¹¹;
R⁸ is hydrogen or C₁-C₆ alkyl;
or R⁷ and R⁸ are taken together with the nitrogen atom to which they are attached to form a substituted or unsubstituted C₂-C₁₀heterocycloalkyl;
each R⁹ is independently selected from hydrogen and C₁-C₆ alkyl;
each R¹⁰ is independently selected from hydrogen and C₁-C₆ alkyl;
R¹¹ is hydrogen, substituted or unsubstituted C₁-C₆ alkyl, substituted or unsubstituted heteroalkyl, substituted or unsubstituted C₂-C₁₀heterocycloalkyl, —C(═O)R¹², —C(═O)—OR¹², —C(═O)N(R¹²)(R⁸), —C(═O)—SR¹², or —P(═O)(OR⁹)₂;
R¹² is hydrogen, substituted or unsubstituted C₁-C₆ alkyl, substituted or unsubstituted heteroalkyl, substituted or unsubstituted C₁-C₁₀cycloalkyl, substituted or unsubstituted C₂-C₁₀heterocycloalkyl, substituted or unsubstituted phenyl, substituted or unsubstituted heteroaryl, -alkyl-(substituted or unsubstituted phenyl), or -alkyl-(substituted or unsubstituted heteroaryl);
m is 1, 2, 3, 4, 5, or 6;
n is 1, 2, 3, 4, 5, or 6;
p is 1, 2, 3, 4, 5, or 6;
wherein substituted means that the referenced group is substituted with one or more additional groups individually and independently selected from halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, C₁-C₆ alkyl, cycloalkyl, fluoro C₁-C₆ alkyl, heteroalkyl, C₁-C₆ alkoxy, fluoro C₁-C₆ alkoxy, heterocycloalkyl, aryl, heteroaryl, aryloxy, C₁-C₆ alkylthio, arylthio, C₁-C₆ alkylsulfoxide, arylsulfoxide, C₁-C₆ alkylsulfone, and arylsulfone.

2. The compound of claim 1, or a pharmaceutically acceptable salt or solvate thereof, having the structure of Formula Ia: 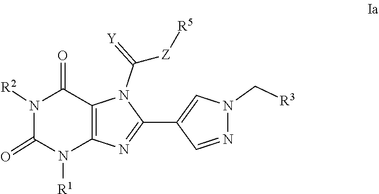
wherein
when Y is O, then Z is absent, S, or —N—(R⁴)—;
when Y is S, then Z is absent, O, S, or —N—(R⁴)—;
R¹ and R² are each independently selected from hydrogen and C₁-C₆ alkyl;
R³ is selected from substituted or unsubstituted phenyl, wherein if R³ is substituted then R³ is substituted with one or more groups selected from halogen, —CN, C₁-C₆ alkyl, and C₁-C₆ fluoroalkyl;
R⁴ is hydrogen or C₁-C₆ alkyl;
R⁵ is hydrogen, R⁷, —C(═O)R⁷, —C(═O)—OR⁷, —C(═O)N(R⁷)(R⁸), —C(═O)—SR⁷, —P(═O)(OR⁹)₂, —(C(R¹⁰)₂O)ₘ—R¹¹, —(CH₂CH₂O)ₙ—R¹¹, or —(C(R¹⁰)₂)ₚ—OR¹¹;
R⁷ is substituted or unsubstituted C₁-C₆ alkyl, substituted or unsubstituted hetero C₁-C₆ alkyl, substituted or unsubstituted C₃-C₁₀ cycloalkyl, substituted or unsubstituted heterocycloalkyl, substituted or unsubstituted phenyl, substituted or unsubstituted heteroaryl, —C₁-C₆ alkyl-(substituted or unsubstituted phenyl), —C₁-C₆ alkyl-(substituted or unsubstituted heteroaryl), —C₁-C₆ alkyl-(substituted or unsubstituted C₃-C₁₀ cycloalkyl), or —C₁-C₆ alkyl-(substituted or unsubstituted heterocycloalkyl);
R⁸ is hydrogen or C₁-C₆ alkyl;
or R⁷ and R⁸ are taken together with the nitrogen atom to which they are attached to form a substituted or unsubstituted heterocycloalkyl;
each R⁹ is independently selected from hydrogen and C₁-C₆ alkyl;
each R¹⁰ is independently selected from hydrogen and C₁-C₆ alkyl;
R¹¹ is hydrogen, substituted or unsubstituted C₁-C₆ alkyl, substituted or unsubstituted heteroalkyl, substituted or unsubstituted heterocycloalkyl, —C(═O)R¹², —C(═O)—OR¹², —C(═O)N(R¹²)(R⁸), —C(═O)—SR¹², or —P(═O)(OR⁹)₂;
R¹² is hydrogen, substituted or unsubstituted C₁-C₆ alkyl, substituted or unsubstituted heteroalkyl, substituted or unsubstituted C₃-C₁₀ cycloalkyl, substituted or unsubstituted heterocycloalkyl, substituted or unsubstituted phenyl, substituted or unsubstituted heteroaryl, —C₁-C₆ alkyl-(substituted or unsubstituted phenyl), or —C₁-C₆ alkyl-(substituted or unsubstituted heteroaryl);
m is 1, 2, 3, 4, 5, or 6;
n is 1, 2, 3, 4, 5, or 6;
p is 1, 2, 3, 4, 5, or 6;
wherein substituted means that the referenced group is substituted with one or more additional groups individually and independently selected from halogen, —CN, —NH₂, —NH(C₁-C₆alkyl), —N(C₁-C₆alkyl)₂, —OH, —CO₂H, —CO₂—C₁-C₆alkyl, —C(═O)NH₂, —C(═O)NH(C₁-C₆ alkyl), —C(═O)N(C₁-C₆ alkyl)₂, —S(═O)₂NH₂, —S(═O)₂NH(C₁-C₆ alkyl), —S(═O)₂N(C₁-C₆ alkyl)₂, C₁-C₆ alkyl, fluoroC₁-C₆ alkyl, C₁-C₆ alkoxy, fluoroC₁-C₆ alkoxy, C₁-C₆ alkylthio, C₁-C₆ alkylsulfoxide, and C₁-C₆ alkylsulfone.

3. The compound of claim 1 of claim 2, or a pharmaceutically acceptable salt or solvate thereof, having the structure of Formula IIa or IIb:

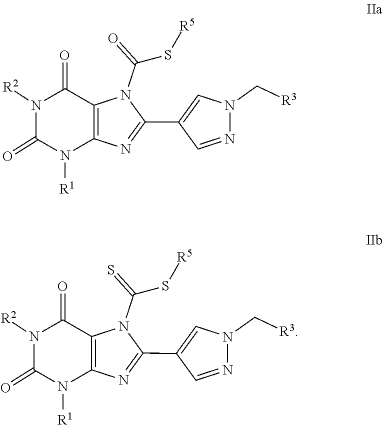

4. The compound of claim 1 or claim 2, or a pharmaceutically acceptable salt or solvate thereof, having the structure of Formula IIIa or IIIb:

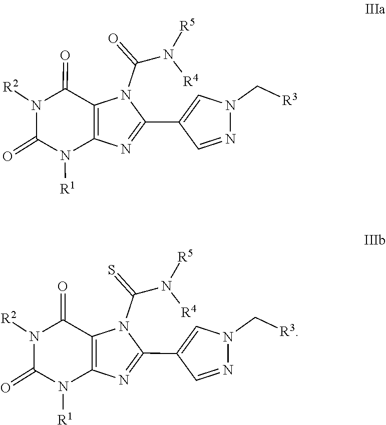

5. The compound of claim 1 or claim 2, or a pharmaceutically acceptable salt or solvate thereof, having the structure of Formula IVa or IVb:

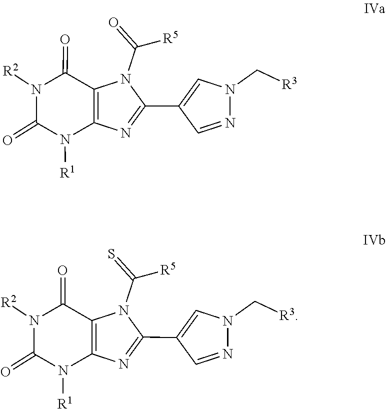

6. The compound of claim 1 or claim 2, or a pharmaceutically acceptable salt or solvate thereof, having the structure of Formula Va or Vb:

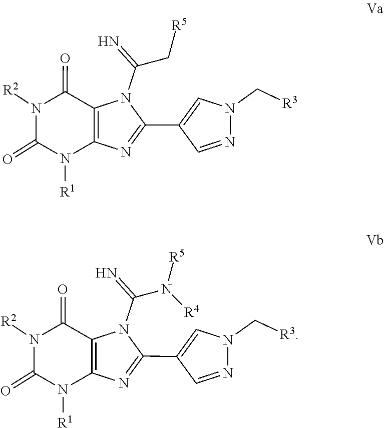

7. The compound of claim 1 or claim 2, or a pharmaceutically acceptable salt or solvate thereof, having the structure of Formula Via or VIb:

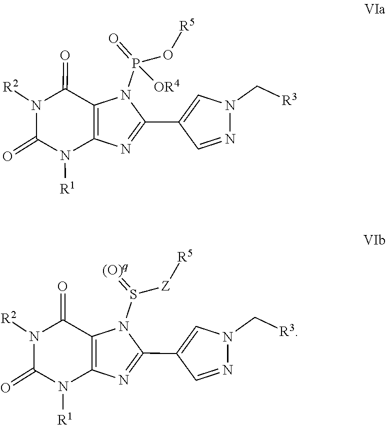

8. The compound of any one of claims 1-7, or a pharmaceutically acceptable salt or solvate thereof, wherein R¹ is methyl, ethyl, or n-propyl.

9. The compound of any one of claims 1-8, or a pharmaceutically acceptable salt or solvate thereof, wherein R² is methyl, ethyl, or n-propyl.

10. The compound of any one of claims 1-9, or a pharmaceutically acceptable salt or solvate thereof, wherein R³ substituted or unsubstituted phenyl, wherein if R³ is substituted then R³ is substituted with one or more groups selected from halogen and C₁-C₆ fluoroalkyl.

11. The compound of any one of claims 1-10, or a pharmaceutically acceptable salt or solvate thereof, wherein R³ is phenyl substituted with C₁-C₆ fluoroalkyl.

12. The compound of any one of claims 1, 2, 4, and 6-11, or a pharmaceutically acceptable salt or solvate thereof, wherein each R⁴ is independently C₁-C₆ alkyl.

13. The compound of any one of claims 1-12 or a pharmaceutically acceptable salt or solvate thereof, wherein R⁵ is —C(═O)R⁷, —C(═O)—OR⁷, —C(═O)N(R⁷)(R⁸), —C(═O)—SR⁷, —P(=O)(OR⁹)₂, —(C(R¹⁰)₂O)ₘ—R¹¹, —(CH₂CH₂O)ₙ—R¹¹, or —(C(R¹⁰)₂)ₚ—OR¹¹.

14. The compound of any one of claims 1-12, or a pharmaceutically acceptable salt or solvate thereof, wherein R⁵ is substituted or unsubstituted C₁-C₆ alkyl, substituted or unsubstituted hetero C₁-C₆ alkyl, substituted or unsubstituted C₃-C₁₀ cycloalkyl, substituted or unsubstituted heterocycloalkyl, substituted or unsubstituted phenyl, substituted or unsubstituted heteroaryl, —C₁-C₆ alkyl-(substituted or unsubstituted phenyl), —C₁-C₆ alkyl-(substituted or unsubstituted heteroaryl), —C₁-C₆ alkyl-(substituted or unsubstituted C₃-C₁₀ cycloalkyl), or —C₁-C₆ alkyl-(substituted or unsubstituted heterocycloalkyl).

15. The compound of any one of claims 1-12, or a pharmaceutically acceptable salt or solvate thereof, wherein R⁵ is substituted or unsubstituted C₁-C₆ alkyl

16. The compound of any one of claims 1-12, or a pharmaceutically acceptable salt or solvate thereof, wherein R⁵ is methyl, ethyl, n-propyl, isopropyl, n-butyl, or isobutyl.

17. A compound, or a pharmaceutically acceptable salt or solvate thereof, selected from:

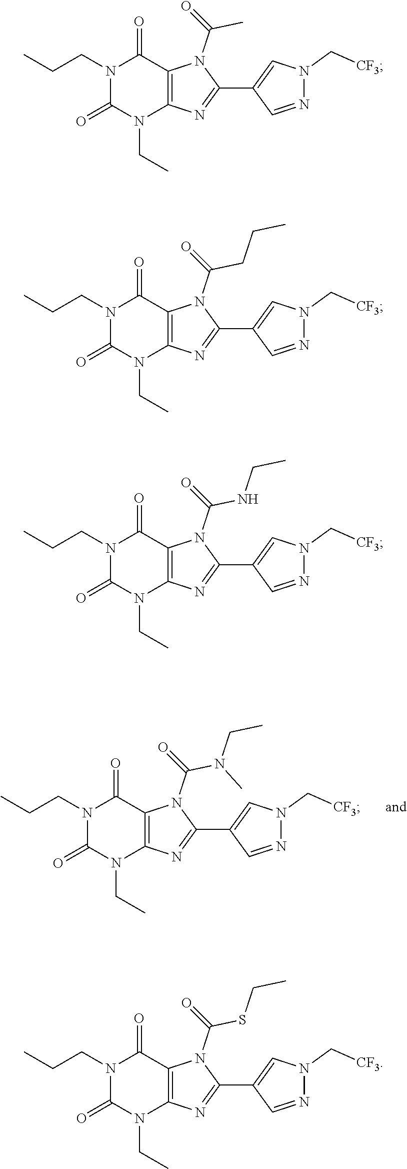 [SMILES: CCCn1c(=O)c2c(nc(-c3cnn(CC(F)(F)F)c3)n2C(C)=O)n(CC)c1=O ; CCCC(=O)n1c(-c2cnn(CC(F)(F)F)c2)nc2c1c(=O)n(CCC)c(=O)n2CC ; CCCn1c(=O)c2c(nc(-c3cnn(CC(F)(F)F)c3)n2C(=O)NCC)n(CC)c1=O ; CCCn1c(=O)c2c(nc(-c3cnn(CC(F)(F)F)c3)n2C(=O)N(C)CC)n(CC)c1=O ; CCCn1c(=O)c2c(nc(-c3cnn(CC(F)(F)F)c3)n2C(=O)SCC)n(CC)c1=O]

18. A compound of Formula A, or a pharmaceutically acceptable salt or solvate thereof, 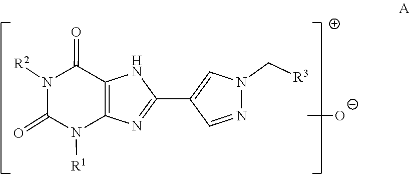
wherein
R¹ and R² are each independently selected from hydrogen and substituted or unsubstituted C₁-C₆alkyl; and
R³ is selected from substituted or unsubstituted phenyl and substituted or unsubstituted heteroaryl, wherein if R³ is substituted then R³ is substituted with one or more groups selected from halogen, —CN, —OH, C₁-C₄alkyl, C₂-C₄alkenyl, C₂-C₄alkynyl, C₁-C₄alkoxy, C₁-C₄fluoroalkyl, C₁-C₄fluoroalkoxy, and substituted or unsubstituted C₁-C₄heteroalkyl.

19. The compound of claim 18, or a pharmaceutically acceptable salt or solvate thereof, having the structure:

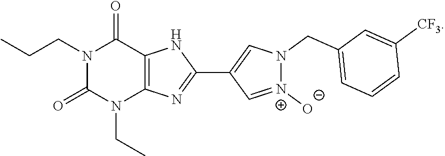 [SMILES: CCCn1c(=O)c2[nH]c(-c3cn(Cc4cccc(C(F)(F)F)c4)[n+]([O-])c3)nc2n(CC)c1=O]

20. A compound of Formula B, or a pharmaceutically acceptable salt or solvate thereof 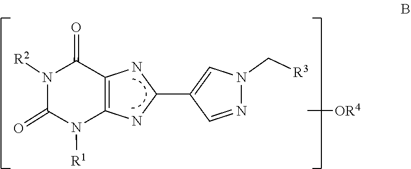
wherein
the dotted line - - - indicates that the referenced ring is aromatic;
R¹ and R² are each independently selected from hydrogen and substituted or unsubstituted C₁-C₆ alkyl;
R³ is selected from substituted or unsubstituted phenyl and substituted or unsubstituted heteroaryl, wherein if R³ is substituted then R³ is substituted with one or more groups selected from halogen, —CN, —OH, C₁-C₄alkyl, C₂-C₄alkenyl, C₂-C₄alkynyl, C₁-C₄alkoxy, C₁-C₄fluoroalkyl, C₁-C₄fluoroalkoxy, and substituted or unsubstituted C₁-C₄heteroalkyl; and
R⁴ is hydrogen, or C₁-C₆ alkyl.

21. The compound of claim 20, or a pharmaceutically acceptable salt or solvate thereof, selected from:

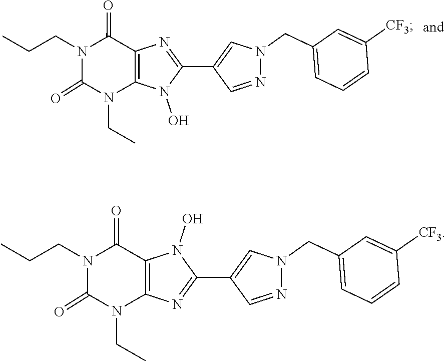 [SMILES: CCCn1c(=O)c2nc(-c3cnn(Cc4cccc(C(F)(F)F)c4)c3)n(O)c2n(CC)c1=O ; "and" ; CCCn1c(=O)c2c(nc(-c3cnn(Cc4cccc(C(F)(F)F)c4)c3)n2O)n(CC)c1=O]

22. A pharmaceutical composition, comprising a compound of any one of claims 1-21, or any pharmaceutically acceptable salt or solvate thereof; and at least one pharmaceutically acceptable excipient.

23. The pharmaceutical composition of claim 22, wherein the pharmaceutical composition is formulated for administration to a mammal by oral administration, intravenous administration, or subcutaneous administration.

24. A method of modulating the A_{2B} adenosine receptor in a mammal comprising administering to the mammal a compound of any one of claims 1-21, or a pharmaceutically acceptable salt or solvate thereof.

25. A method of treating a disease or disorder in a mammal comprising administering to the mammal in need thereof a therapeutically effective amount of a compound of any one of claims 1-21, or a pharmaceutically acceptable salt or solvate thereof, wherein the condition is selected from the group consisting of cardiovascular diseases, fibrosis, neurological disorders, type I hypersensitivity disorders, chronic and acute liver discases, lung diseases, renal discases, diabetes, obesity, and cancer.

26. The method of claim 25, wherein the condition is cancer.

27. The method of claim 26, wherein the cancer is selected from bladder cancer, colon cancer, brain cancer, breast cancer, endometrial cancer, heart cancer, kidney cancer, lung cancer, liver cancer, uterine cancer, blood and lymphatic cancer, ovarian cancer, pancreatic cancer, prostate cancer, thyroid cancer, gastric cancer, rectal cancer, urothelial cancer, testes cancer, cervical cancer, vaginal cancer, vulvar cancer, head and neck cancer, and skin cancer.

28. The method of claim 26, wherein the cancer is a hormone-related cancer.

29. The method of claim 28, wherein the hormone-related cancer is breast cancer, endometrial cancer, ovarian cancer, prostate cancer, testicular cancer, thyroid cancer or osteosarcoma.

30. The method of claim 28, wherein the hormone-related cancer is metastatic castration resistant prostate cancer.

31. The method of claim 28, wherein the hormone-related cancer is breast cancer.

32. The method of any one of claims 24-31, further comprising administration of a second agent.

33. The method of claim 32, wherein the second agent is an anti-PD-1 agent or an anti-PDL-1 agent.
